(19) United States
(12) Patent Application Publication
(10) Pub. No.: US 2020/0261383 A1
(43) Pub. Date: Aug. 20, 2020

(54) PHARMACEUTICAL COMPOSITIONS

(71) Applicant: KALVISTA PHARMACEUTICALS LIMITED, Wiltshire (GB)

(72) Inventors: Gary COOK, Westford, MA (US); Sally Louise MARSH, Salisbury (GB); Stephen John PETHEN, Salisbury (GB); Michael Bryan ROE, Salisbury (GB); Christopher Martyn YEA, Salisbury (GB)

(21) Appl. No.: 16/637,953

(22) PCT Filed: Aug. 13, 2018
(86) PCT No.: PCT/GB2018/052292

§ 371 Date: Feb. 10, 2020

Related U.S. Application Data
(60) Provisional application No. 62/544,346, filed on Aug. 11, 2017.

(30) Foreign Application Priority Data
Aug. 25, 2017 (GB) 1713660.7

(51) Int. Cl. A61K 31/166; A61K 47/26; A61K 9/08; A61P 27/02

(52) U.S. Cl. CPC A61K 31/166; A61P 27/02; A61K 9/08; A61K 47/26

(57) ABSTRACT

The invention relates to aqueous solution pharmaceutical compositions comprising at least one non-ionic tonicity agent, at least one buffer and an active ingredient, wherein said active ingredient is a compound of formula I, wherein R¹-R⁹ are defined herein; processes for preparing these compositions and their use in medicine, especially their use in the treatment of ocular diseases.

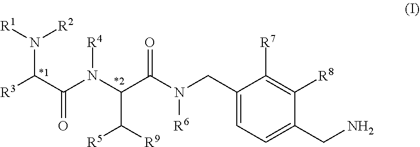

### TECHNICAL FIELD

The present invention relates to aqueous solution pharmaceutical compositions of small molecule plasma kallikrein inhibitors, processes for preparing these compositions and their use in medicine, especially their use in the treatment of ocular diseases.

### BACKGROUND OF THE INVENTION

The plasma kallikrein-kinin system is a system of blood proteins that plays a role in inflammation, blood pressure control, coagulation and pain. The plasma kallikrein-kinin system is abnormally abundant in patients with advanced diabetic macular edema. It has recently been published that plasma kallikrein contributes to retinal vascular dysfunctions in diabetic rats (A. Clermont et al. “Plasma kallikrein mediates retinal vascular dysfunction and induces retinal thickening in diabetic rats” Diabetes, 2011, 60, p1590-98). Furthermore, administration of the plasma kallikrein inhibitor ASP-440 ameliorated both retinal vascular permeability and retinal blood flow abnormalities in diabetic rats. Therefore, a plasma kallikrein inhibitor should have utility as a treatment to reduce retinal vascular permeability associated with diabetic retinopathy and diabetic macular edema. Other complications of diabetes such as cerebral haemorrhage, nephropathy, cardiomyopathy and neuropathy, all of which have associations with plasma kallikrein may also be considered as targets for a plasma kallikrein inhibitor.

Synthetic and small molecule plasma kallikrein inhibitors have been described previously, for example by Garrett et al. (“Peptide aldehyde . . . .” J. Peptide Res. 52, p62-71 (1998)), T. Griesbacher et al. (“Involvement of tissue kallikrein but not plasma kallikrein in the development of symptoms mediated by endogenous kinins in acute pancreatitis in rats” British Journal of Pharmacology 137, p692-700 (2002)), Evans (“Selective dipeptide inhibitors of kallikrein” WO03/076458), Szelke et al. (“Kininogenase inhibitors” WO92/04371), D. M. Evans et al. (Immunolpharmacology, 32, p115-116 (1996)), Szelke et al. (“Kininogen inhibitors” WO95/07921), Antonsson et al. (“New peptides derivatives” WO94/29335), J. Corte et al. (“Six membered heterocycles useful as serine protease inhibitors” WO2005/123680), J. Sturzbecher et al. (Brazilian J. Med. Biol. Res 27, p1929-34 (1994)), Kettner et al. (US 5,187,157), N. Teno et al. (Chem. Pharm. Bull. 41, p1079-1090 (1993)), W. B. Young et al. (“Small molecule inhibitors of plasma kallikrein” Bioorg. Med. Chem. Letts. 16, p2034-2036 (2006)), Okada et al. (“Development of potent and selective plasmin and plasma kallikrein inhibitors and studies on the structure-activity relationship” Chem. Pharm. Bull. 48, p1964-72 (2000)), Steinmetzer et al. (“Trypsin-like serine protease inhibitors and their preparation and use” WO08/049595), Zhang et al. (“Discovery of highly potent small molecule kallikrein inhibitors” Medicinal Chemistry 2, p545-553 (2006)), Sinha et al. (“Inhibitors of plasma kallikrein” WO08/016883), Shigenaga et al. (“Plasma Kallikrein Inhibitors” WO2011/118672), and Kolte et al. (“Biochemical characterization of a novel high-affinity and specific kallikrein inhibitor”, British Journal of Pharmacology (2011), 162(7), 1639-1649). Also, Steinmetzer et al. (“Serine protease inhibitors” WO2012/004678) describes cyclized peptide analogs which are inhibitors of human plasmin and plasma kallikrein.

To date, the only selective plasma kallikrein inhibitor approved for medical use is Ecallantide. Ecallantide is formulated as a solution for injection. It is a large protein plasma kallikrein inhibitor that presents a risk of anaphylactic reactions. Other plasma kallikrein inhibitors known in the art are generally small molecules, some of which include highly polar and ionisable functional groups, such as guanidines or amidines. Recently, plasma kallikrein inhibitors that do not feature guanidine or amidine functionalities have been reported. For example Brandi et al. (“N-((6-amino-pyridin-3-yl)methyl)-heteroaryl-carboxamides as inhibitors of plasma kallikrein” WO2012/017020), Evans et al. (“Benzylamine derivatives as inhibitors of plasma kallikrein” WO2013/005045), Allan et al. (“Benzylamine derivatives” WO2014/108679), and Davie et al. (“Heterocyclic derivates” WO2014/188211).

Intravitreal injection of plasma kallikrein inhibitors is known (for example, see Evans et al. WO2013/005045) and allows the plasma kallikrein inhibitor to be delivered directly to the ocular tissues. However, small molecules dosed as solutions and administered by intravitreal injection are typically cleared from the vitreous within hours (for example, see “Review: Practical Issues in Intravitreal Drug Delivery”, Journal of Ocular Pharmacology and Therapeutics, Volume 17, Number 4, 2001, p393-401, David Maurice and “Prediction of Vitreal Half-Life Based on Drug Physiochemical Properties: Quantitative Structure-Pharmacokinetic Relationships (QSPKR)”, Pharmaceutical Research, Volume 26, Number 5, 2009, p1236-1260, Chandrasekar Durairaj et al.).

The previous reports of plasma kallikrein inhibitors do not provide evidence of extended duration of action, for example greater than 7 days, from an aqueous solution formulation administered by intravitreal injection. For example, Evans presents pharmacokinetic data for one compound following intravitreal injection to rabbits. However, the disclosed data is limited to only 7 days post dosing; no data past this time point is described.

Intravitreal injection is an invasive procedure, and therefore reduced clearance and an extended duration of action are desirable to reduce the period required between injections. Cook et al. (“Pharmaceutical compositions” WO2014/108685) discloses compositions containing suspended plasma kallikrein inhibitors with relatively long dissolution times, thus providing a relatively long period of action. However, a problem with pharmaceutical compositions containing suspended actives is that additional manufacturing steps are required, such as reducing the particle size of the active ingredient and controlling the particle size distribution of the active ingredient. There is also a risk of non-homogeneity of the suspension in the formulation.

Accordingly, there is a need for a pharmaceutical composition comprising a plasma kallikrein inhibitor that has a long duration of action, and does not have the disadvantages associated with a suspension of active ingredient. Moreover, there is a need for a pharmaceutical composition that is suitable for injection into the eye, and has a long duration of action in the ocular tissues, particularly the retina.

### SUMMARY OF THE INVENTION

It is an object of the present invention to provide a pharmaceutical composition of a plasma kallikrein inhibitor that may be administered parenterally, and in particular administered by intravitreal injection. It is a further object of the present invention to provide a pharmaceutical composition of a plasma kallikrein inhibitor with a relatively long period of action, particularly in the ocular tissues, resulting in the compositions being administered less frequently than would otherwise be necessary while maintaining sufficient levels of the plasma kallikrein inhibitor in vivo to provide the intended clinical effect.

Surprisingly, it has been found that the pharmaceutical composition of the present invention, which comprises a plasma kallikrein inhibitor and is in the form of an aqueous solution, is able to provide a relatively long period of action when administered by intravitreal injection. The levels of the active ingredient in the retina after administering the pharmaceutical composition were found to be particularly high.

The aqueous solution pharmaceutical compositions of the present invention are advantageous over the aqueous suspension pharmaceutical compositions of Cook et al. since the aqueous solutions avoid the need for additional manufacturing steps associated with suspended actives, such as reducing the particle size of the active ingredient and controlling the particle size distribution of the active ingredient, while maintaining the long period of action of the plasma kallikrein inhibitor. A further advantage of aqueous solution pharmaceutical compositions of the present invention over the aqueous suspension pharmaceutical compositions of Cook et al. is the much reduced risk of non-homogeneity of a solution compared to a suspension.

In the present application, the aforesaid plasma kallikrein inhibitor hereinafter refers to a compound of formula I as defined below and as disclosed in Evans et al. (“Benzylamine derivatives as inhibitors of plasma kallikrein” WO2013/005045).

Intravitreal injection of the compositions of the present invention results in slow elimination of the active ingredient from the vitreous humor. Moreover, particularly high concentrations of the active ingredient in the retina and in the choroid are observed, which confirms that the active ingredient reaches the posterior ocular tissues.

It is therefore a further object of the invention to provide a composition with improved patient compliance, by reducing the frequency at which the composition needs to be administered to a subject.

The present invention relates to a pharmaceutical composition in the form of an aqueous solution for parenteral administration comprising at least one non-ionic tonicity agent, at least one buffer and an active ingredient, wherein said active ingredient is a compound of formula I

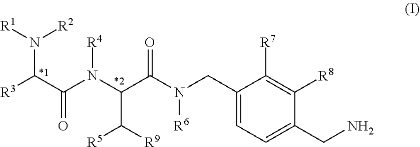

wherein:
R¹ is selected from H, alkyl, —COalkyl, —COaryl, —COheteroaryl, —CO₂alkyl, —(CH₂)ₐ0H, —(CH₂)_{b}COOR¹⁰, —(CH₂)_{c}CONH₂, —SO₂alkyl and —SO₂aryl;
R² is selected from H and alkyl;
R³ is selected from H, alkyl, —(CH₂)_{d}aryl, —(CH₂)ₑheteroaryl, —(CH₂)_{f}cycloalkyl, —(CH₂)_{g}heterocycloalkyl, —CH(cycloalkyl)₂ and —CH(heterocycloalkyl)₂;
R⁴ and R⁶ are independently selected from H and alkyl;
R⁵ is selected from H, alkyl, alkoxy and OH;
or R⁴ and R⁵, together with the atoms to which they are attached, may join to form a 5- or 6-memebered azacycloalkyl structure;
R⁷ and R⁸ are independently selected from H, alkyl, alkoxy, CN and halo;
R⁹ is aryl or heteroaryl;
R¹⁰ is H or alkyl;
a, b, c, d, e, f and g are independently 1, 2 or 3;
*1 and *2 denote chiral centres;
alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1 or 2 substituents independently selected from (C₃-C₁₀)cycloalkyl, (C₁-C₆)alkoxy, OH, CN, CF₃, COOR¹¹, fluoro and NR¹¹R¹²;
cycloalkyl is a mono- or bi-cyclic saturated hydrocarbon of between 3 and 10 carbon atoms; cycloalkyl may optionally be fused to an aryl group;
heterocycloalkyl is a C-linked or N-linked 3 to 10 membered saturated, mono- or bi-cyclic ring, wherein said heterocycloalkyl ring contains, where possible, 1, 2 or 3 heteroatoms independently selected from N, NR¹¹ and O;
alkoxy is a linear O-linked hydrocarbon of between 1 and 6 carbon atoms (C₁-C₆) or a branched O-linked hydrocarbon of between 3 and 6 carbon atoms (C₃-C₆); alkoxy may optionally be substituted with 1 or 2 substituents independently selected from (C₃-C₁₀)cycloalkyl, OH, CN, CF₃, COOR¹¹, fluoro and NR¹¹R¹²;
aryl is phenyl, biphenyl or naphthyl; aryl may be optionally substituted with up to 5 substituents independently selected from alkyl, alkoxy, OH, halo, CN, COOR¹¹, CF₃ and NR¹¹R¹²;
heteroaryl is a 5, 6, 9 or 10 membered mono- or bi-cyclic aromatic ring, containing, where possible, 1, 2 or 3 ring members independently selected from N, NR¹¹, S and O; heteroaryl may be optionally substituted with 1, 2 or 3 substituents independently selected from alkyl, alkoxy, OH, halo, CN, COOR¹¹, CF₃ and NR¹¹R¹²;
R¹¹ and R¹² are independently selected from H and alkyl;

and tautomers, isomers, stereoisomers (including enantiomers, diastereoisomers and racemic and scalemic mixtures thereof), and pharmaceutically acceptable salts thereof.

The present invention further relates to methods for treating a disease or condition mediated by plasma kallikrein comprising parenteral administration of a pharmaceutical composition of the invention to a mammal.

The disease or condition mediated by plasma kallikrein may be selected from impaired visual acuity, diabetic retinopathy, retinal vascular permeability associated with diabetic retinopathy, diabetic macular edema, retinal vascular occlusion, hereditary angioedema, diabetes, pancreatitis, cerebral haemorrhage, cerebral haemorrhage in hyperglycemic patients, nephropathy, cardiomyopathy, neuropathy, inflammatory bowel disease, arthritis, inflammation, septic shock, hypotension, cancer, adult respiratory distress syndrome, disseminated intravascular coagulation, blood coagulation during cardiopulmonary bypass surgery and bleeding from post-operative surgery.

Preferably, the disease or condition mediated by plasma kallikrein is selected from impaired visual acuity, diabetic retinopathy, retinal vascular permeability associated with diabetic retinopathy, diabetic macular edema, and retinal vascular occlusion. More preferably, the disease or condition mediated by plasma kallikrein is retinal vascular permeability associated with diabetic retinopathy or diabetic macular edema.

### BRIEF DESCRIPTION OF DRAWINGS

FIG. 1 Composite ocular tissue concentration-time profile following single bilateral intravitreal injection of Compound 1 on Day 1. Data are expressed as individual eye data from one animal per sample time point.

FIG. 2 Mean ocular tissue concentration-time profile following bilateral intravitreal injection of Compound 1 to rabbits on Day 1, 29 and 57. Data are expressed as mean data from one animal per sample time point.

FIG. 3 Mean ocular tissue concentration-time profile (Day 58-99) following single bilateral intravitreal injection of Compound 1 to monkeys on Day 1, 29 and 57. Data are expressed as mean data from one animal per sample time point.

### DETAILED DESCRIPTION OF THE INVENTION

Active ingredient The pharmaceutical compositions of the invention are aqueous solutions. Preferably, the pharmaceutical composition of the invention meets the requirements of USP <788> (Particulate matter in injections) for a small-volume injection with a container volume of 2 mL when measured using the microscopic particle count test. The acceptance limits provided in USP <788> for a small-volume injection using the microscopic particle count test are that the number of particles present (actual or calculated) in each discrete unit tested or in each pooled sample tested does not exceed 3000 per container equal to or greater than 10 μm, and does not exceed 300 per container equal to or greater than 25 μm.

More preferably, the pharmaceutical composition of the invention meets the requirements of USP <788> (Particulate matter in injections) for a large-volume injection when measured using the microscopic particle count test. The acceptance limits provided in USP <788> for a large-volume injection using the microscopic particle count test are that the number of particles present (actual or calculated) in each discrete unit tested or in each pooled sample tested does not exceed 12 per mL equal to or greater than 10 μm, and does not exceed 2 per mL equal to or greater than 25 μm.

More preferably, the pharmaceutical composition of the invention meets the requirements of USP <789> (Particulate matter in ophthalmic solutions) when measured using the microscopic particle count test. The acceptance limits provided in USP <789> using the microscopic particle count test are that the average number of particles present in the units tested does not exceed 50 per mL equal to or greater than 10 μm, and does not exceed 5 per mL equal to or greater than 25 μm, and does not exceed 2 per mL equal to or greater than 50 μm.

The references to USP <788> and USP <789> herein refer to USP <788> and USP <789> in United States Pharmacopeia (USP) 37, NF 32.

The compositions of the invention are aqueous, but can be pre-formulated as a sterile, non-aqueous solution or in a dried form which can be subsequently reconstituted with a suitable aqueous vehicle (e.g. sterile, pyrogen-free water). In some embodiments the composition may be provided as a bulk solution which is further diluted, for example with sterile, pyrogen-free water, prior to use.

The compositions of the invention may be hypotonic, isotonic or hypertonic. The compositions of the invention typically have an osmolality of from about 250 to about 350 mOsmol/kg. For example, the compositions may have an osmolality of 250, 260, 270, 280, 290, 300, 310, 320, 330, 340 or 350 mOsmol/kg.

The compositions will typically be at a pH of from about 2 to about 10, e.g. pH 2, 3, 4, 5, 6, 7, 8, 9 or 10. Preferably, the compositions will be at a pH of from about 4 to about 8, for example at a pH of from about 5 to about 7.5. More preferably, the compositions will be at a pH of from about 5 to about 6.

The composition of the invention includes a plasma kallikrein inhibitor of formula I as active ingredient. Typically, the active ingredient is present in the composition at a concentration of from about 5 μg/mL to about 500 μg/mL, or from about 5 μg/mL to about 300 μg/mL, or from about 30 μg/mL to about 300 μg/mL, or from about 5 μg/mL to about 200 μg/mL, or from about 10 μg/mL to about 200 μg/mL. In a preferred embodiment the active ingredient is present in the composition at a concentration of from about 10 μg/mL to about 200 μg/mL. Typically, the active ingredient is present in the composition at a concentration of about 5 μg/mL, 10 μg/mL, 20 μg/mL, 30 μg/mL, 40 μg/mL, 50 μg/mL, 60 μg/mL, 80 μg/mL, 100 μg/mL, 150 μg/mL, or 200 ug/mL. The concentrations specified refer to the concentration of the free base of the plasma kallikrein inhibitor of formula I in the composition. The free base of the plasma kallikrein inhibitor of formula I has the structure depicted in formula I.

The active ingredient is a plasma kallikrein inhibitor of formula I

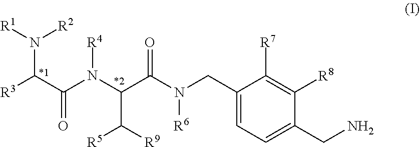

wherein:
R¹ is selected from H, alkyl, —COalkyl, —COaryl, —COheteroaryl, —CO₂alkyl, —(CH₂)ₐOH, —(CH₂)_{b}COOR¹⁰, —(CH₂)_{c}CONH₂, —SO₂alkyl and —SO₂aryl;
R² is selected from H and alkyl;
R³ is selected from H, alkyl, —(CH₂)_{d}aryl, —(CH₂)ₑheteroaryl, —(CH₂)_{f}cycloalkyl, —(CH₂)_{g}heterocycloalkyl, —CH(cycloalkyl)₂ and —CH(heterocycloalkyl)₂;
R⁴ and R⁶ are independently selected from H and alkyl;
R⁵ is selected from H, alkyl, alkoxy and OH;
or R⁴ and R⁵, together with the atoms to which they are attached, may join to form a 5- or 6-memebered azacycloalkyl structure;
R⁷ and R⁸ are independently selected from H, alkyl, alkoxy, CN and halo;
R⁹ is aryl or heteroaryl;
R¹⁰ is H or alkyl;
a, b, c, d, e, f and g are independently 1, 2 or 3;
*1 and *2 denote chiral centres;
alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1 or 2 substituents independently selected from (C₃-C₁₀)cycloalkyl, (C₁-C₆)alkoxy, OH, CN, CF₃, COOR¹¹, fluoro and NR¹¹R¹²;
cycloalkyl is a mono- or bi-cyclic saturated hydrocarbon of between 3 and 10 carbon atoms; cycloalkyl may optionally be fused to an aryl group;
heterocycloalkyl is a C-linked or N-linked 3 to 10 membered saturated, mono- or bi-cyclic ring, wherein said heterocycloalkyl ring contains, where possible, 1, 2 or 3 heteroatoms independently selected from N, NR¹¹ and O;
alkoxy is a linear O-linked hydrocarbon of between 1 and 6 carbon atoms (C₁-C₆) or a branched O-linked hydrocarbon of between 3 and 6 carbon atoms (C₃-C₆); alkoxy may optionally be substituted with 1 or 2 substituents independently selected from (C₃-C₁₀)cycloalkyl, OH, CN, CF₃, COOR¹¹, fluoro and NR^{IIR12;}
aryl is phenyl, biphenyl or naphthyl; aryl may be optionally substituted with up to 5 substituents independently selected from alkyl, alkoxy, OH, halo, CN, COOR¹¹, CF₃ and NR¹¹R¹²;
heteroaryl is a 5, 6, 9 or 10 membered mono- or bi-cyclic aromatic ring, containing, where possible, 1, 2 or 3 ring members independently selected from N, NR¹¹, S and O; heteroaryl may be optionally substituted with 1, 2 or 3 substituents independently selected from alkyl, alkoxy, OH, halo, CN, COOR¹¹, CF₃ and NR¹¹R¹²;
R¹¹ and R¹² are independently selected from H and alkyl;
and tautomers, isomers, stereoisomers (including enantiomers, diastereoisomers and racemic and scalemic mixtures thereof), and pharmaceutically acceptable salts thereof.

In some embodiments, the plasma kallikrein inhibitor is a compound of formula I wherein:

R¹ is selected from H, alkyl, —COalkyl, —COaryl, —CO₂alkyl, —CH₂CH₂OH, —CH₂COOR¹°, —CH₂CONH₂, —SO₂alkyl and —SO₂aryl;

R² is selected from H and alkyl;
R³ is selected from alkyl, —CH₂aryl, —CH₂cycloalkyl and —CH(cycloalkyl)₂;
R⁴ and R⁶ are independently selected from H and alkyl;
R⁵ is selected from H, alkyl, and OH;
or R⁴ and R⁵, together with the atoms to which they are attached, may join to form a 5- or 6-memebered azacycloalkyl structure;
R⁷ and R⁸ are independently selected from H, F, and Cl;
R⁹ is aryl;
R¹⁰ is H or alkyl;
*1 and *2 denote chiral centres;
alkyl is a linear saturated hydrocarbon having up to 6 carbon atoms (C₁-C₆) or a branched saturated hydrocarbon of between 3 and 6 carbon atoms (C₃-C₆); alkyl may optionally be substituted with 1 or 2 substituents independently selected from (C₃-C₁₀)cycloalkyl, (C₁-C₆)alkoxy, OH, CN, CF₃, COOR¹¹, fluoro and NR¹¹R¹²;
cycloalkyl is a mono- or bi-cyclic saturated hydrocarbon of between 3 and 10 carbon atoms;
alkoxy is a linear O-linked hydrocarbon of between 1 and 6 carbon atoms (C₁-C₆) or a branched O-linked hydrocarbon of between 3 and 6 carbon atoms (C₃-C₆); alkoxy may optionally be substituted with 1 or 2 substituents independently selected from (C₃-C₁₀)cycloalkyl, OH, CN, CF₃, COOR¹¹, fluoro and NR¹¹R¹²;
aryl is phenyl, biphenyl or naphthyl; aryl may be optionally substituted with up to 5 substituents independently selected from alkyl, alkoxy, OH, halo, CN, COOR¹¹, CF₃ and NR¹¹R¹²;
R¹¹ and R¹² are independently selected from H and alkyl;

and tautomers, isomers, stereoisomers (including enantiomers, diastereoisomers and racemic and scalemic mixtures thereof), and pharmaceutically acceptable salts thereof.

In an embodiment of the invention, R¹ is selected from H, alkyl, —COalkyl, —COaryl, —(CH₂)ₐOH, —(CH₂)_{b}COOR¹⁰, —(CH₂)_{c}CONH₂, —SO₂alkyl and —SO₂aryl.

In an embodiment of the invention, R¹ is selected from H, alkyl, —COalkyl, —COaryl, —(CH₂)ₐOH, —CH₂COOR¹⁰, —CH₂CONH₂, —SO₂alkyl and —SO₂aryl; wherein a is 1 or 2.

In an embodiment of the invention, R¹ is selected from H, —COaryl, —COalkyl, —CH₂COOH, —SO₂Ph and —SO₂CH₃.

In an embodiment of the invention, R¹ is selected from H, —COethyl, methyl, methylsulfonyl, —COphenyl, phenylsulfone, —CH₂COOH, —CO-'propyl, propyl, —CH₂COOCH₃, —CH₂CONH₂, —CH₂CH₂OH and COnaphthyl.

In an embodiment of the invention, R¹ is selected from —COalkyl and COphenyl.

In an embodiment of the invention, R¹ is selected from H, —COaryl, COheteroaryl, —COalkyl, —CH₂COOH, —SO₂Ph and —SO₂CH₃.

In an embodiment of the invention, R¹ is selected from —COalkyl, COheteroaryl and —COaryl.

In an embodiment of the invention, R² is selected from H and methyl.

In an embodiment of the invention, R² is H.

In an embodiment of the invention, R³ is selected from alkyl, —(CH₂)_{d}aryl, —(CH₂)_{f}cycloalkyl, and —CH(cycloalkyl)₂; wherein d and f are, independently, 1 or 2.

In an embodiment of the invention, R³ is selected from alkyl, —CH₂aryl, —CH₂cycloalkyl, and —CH(cycloalkyl)₂.

In an embodiment of the invention, R³ is selected from —CH₂aryl, —CH₂cycloalkyl, and —CH(cycloalkyl)₂.

In an embodiment of the invention, R³ is selected from:

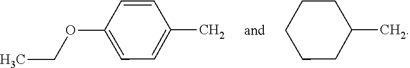

In an embodiment of the invention, R⁴ is selected from H and methyl.

In an embodiment of the invention, R⁴ is H.

In an embodiment of the invention, R⁵ is selected from H, alkyl and OH.

In an embodiment of the invention, R⁵ is selected from H and OH.

In an embodiment of the invention, R⁵ is H.

In an embodiment of the invention, R⁴ and R⁵, together with the atoms to which they are attached, join to form a pyrrolidine moiety.

In an embodiment of the invention, R⁴ and R⁵, together with the atoms to which they are attached, join to form a piperidine moiety.

In an embodiment of the invention, R⁶ is selected from H and methyl.

In an embodiment of the invention, R⁶ is H.

In an embodiment of the invention, R⁷ is selected from H, methyl and halo.

In an embodiment of the invention, R⁷ is selected from H, F and Cl.

In an embodiment of the invention, R⁷ is H.

In an embodiment of the invention, R⁸ is selected from H, methyl and halo.

In an embodiment of the invention, R⁸ is selected from H, F and Cl.

In an embodiment of the invention, R⁸ is selected from H and F.

In an embodiment of the invention, R⁸ is H.

In an embodiment of the invention, R⁹ is aryl.

In an embodiment of the invention, R⁹ is selected from phenyl and naphthyl, wherein phenyl may be optionally substituted with up to 3 substituents independently selected from alkyl, alkoxy, OH, halo, CN, COOR¹¹, CF₃ and NR¹¹R¹².

In an embodiment of the invention, R⁹ is phenyl, wherein phenyl may be optionally substituted with up to 2 substituents independently selected from alkyl, halo and CF₃.

In an embodiment of the invention, R⁹ is selected from phenyl, 1-naphthalene, 2,4-dichlorophenyl, 3,4-dichlorophenyl, 3,4-difluorophenyl, 4-chlorophenyl, 4-trifluoromethylphenyl and 4-ethoxyphenyl.

In an embodiment of the invention, R⁹ is selected from phenyl, heteroaryl and naphthyl, wherein phenyl may be optionally substituted with up to 3 substituents independently selected from alkyl, alkoxy, OH, halo, CN, COOR¹¹, CF₃ and NR¹¹R¹².

In an embodiment of the invention, R⁹ is selected from phenyl, 1-naphthalene, 3,4-dichlorophenyl, 3,4-difluorophenyl, 4-chlorophenyl, 4-fluorophenyl, 3-fluorophenyl, 4-trifluoromethylphenyl, pyrid-3-yl, pyrid-2-yl, pyrid-4-yl, benzothiophen-3-yl, thiophen-2-yl, thiophen-3-yl, indol-3-yl, and thiazol-4y1.

In an embodiment of the invention, R¹⁰ is H or methyl.

In an embodiment of the invention, the stereochemical configuration about chiral centre *1 is R.

In an embodiment of the invention, the stereochemical configuration about chiral centre *2 is S.

In an embodiment of the invention, a is 2 and b, c, d, e, f and g are 1.

In an embodiment of the invention, a is 2 and b, c, d, e, f, g, h, j, l and m are 1.

In an embodiment of the invention, k is 0 or 1.

In preferred embodiments, the active ingredient is a compound selected from:

(S)-N-(4-Aminomethyl-benzyl)-2-[(R)-3-(4-ethoxy-phenyl)-2-propionylamino-propionylamino]-3-phenyl-propionamide;

N-[(R)-1-[(S)-1-(4-Aminomethyl-benzylcarbamoyl)-2-phenyl-ethylcarbamoyl]-2-(4-ethoxy-phenyl)-ethyl]-benzamide;

{(R)-1-[(S)-1 -(4-Aminomethyl-benzylcarbamoyl)-2-phenyl-ethylcarbamoyl]-2-cyclohexyl-ethylamino}-acetic acid;

(S)-N-(4-Aminomethyl-3-fluoro-benzyl)-2-[(R)-3-(4-ethoxy-phenyl)-2-propionylamino-propionylamino]-3-phenyl-propionamide;

(S)-N-(4-Aminomethyl-2-chloro-benzyl)-2-[(R)-3-(4-ethoxy-phenyl)-2-propionylamino-propionylamino]-3-phenyl-propionamide;

(S)-N-(4-Aminomethyl-benzyl)-3-(3,4-dichloro-phenyl)-2-[(R)-3-(4-ethoxy-phenyl)-2-propionylamino-propionylamino]-propionamide;

(S)-N-(4-Aminomethyl-3-chloro-benzyl)-2-[(R)-3-(4-ethoxy-phenyl)-2-propionylamino-propionylamino]-3-phenyl-propionamide;

(S)-N-(4-Aminomethyl-benzyl)-2-{[(R)-3-(4-ethoxy-phenyl)-2-propionylamino-propionyl]-methyl-amino}-3-phenyl-propionamide;

({(R)-1-[(S)-1-(4-Aminomethyl-benzylcarbamoyl)-2-phenyl-ethylcarbamoyl]-2-cyclohexyl-ethyl}-methyl-amino)-acetic acid;

(S)-N-(4-Aminomethyl-3-fluoro-benzyl)-2-[(R)-3-(4-ethoxy-phenyl)-2-propionylamino-propionyl]-methyl-amino)-3-phenyl-propionamide;

N-[(R)-1-{[(S)-1-(4-Aminomethyl-benzylcarbamoyl)-2-phenyl-ethyl]-methyl-carbamoyl}-2-(4-ethoxy-phenyl)-ethyl]-benzamide;

N-[(R)-1-{[(S)-1-(4-Aminomethyl-benzylcarbamoyl)-2-phenyl-ethyl]-methyl-carbamoyl}-2-(4-ethoxy-phenyl)-ethyl]-isobutyramide;

Naphthalene-1-carboxylic acid [(R)-1-[(S)-1-(4-aminomethyl-benzylcarbamoyl)-2-phenyl-ethylcarbamoyl]-2-(4-ethoxy-phenyl)-ethyl]-amide;

N-[(R)-1-[(S)-1-(4-Aminomethyl-benzylcarbamoyl)-2-phenyl-ethylcarbamoyl]-2-(4-ethoxy-phenyl)-ethyl]-4-chloro-benzamide;

N-[(R)-1-[(S)-1-(4-Aminomethyl-benzylcarbamoyl)-2-phenyl-ethylcarbamoyl]-2-(4-ethoxy-phenyl)-ethyl]-2,4-dichloro-benzamide;

N-[(R)-1-[(S)-1-(4-Aminomethyl-benzylcarbamoyl)-2-phenyl-ethylcarbamoyl]-2-(4-ethoxy-phenyl)-ethyl]-3,4-difluoro-benzamide;

(R)-2-Amino-N-[(1S,2S)-1-(4-aminomethyl-benzylcarbamoyl)-2-hydroxy-2-phenyl-ethyl]-3 -(4-ethoxy-phenyl)-propionamide;

N-[(R)-1-[(S)-1-(4-Aminomethyl-benzylcarbamoyl)-2-phenyl-ethylcarbamoyl]-2-(4-ethoxy-phenyl)-ethyl]-nicotinamide;

(2S,3 S)-N-(4-Aminomethyl-benzyl)-2-[(R)-3-(4-ethoxy-phenyl)-2-propionylamino-propionylamino]-3-hydroxy-3-phenyl-propionamide;

N-[(R)-1-[(S)-1-(4-Aminomethyl-benzylcarbamoyl)-2-phenyl-ethylcarbamoyl]-2-(4-ethoxy-phenyl)-ethyl]-isonicotinamide;

Thiophene-3 -carboxylic acid-[(R)-1-[(S)-1-(4-aminomethyl-benzylcarbamoyl)-2-phenyl-ethylcarbamoyl]-2-(4-ethoxy-phenyl)-ethyl]-amide;

Thiophene-2-carboxylic acid [(R)-1-[(S)-1-(4-aminomethyl-benzylcarbamoyl)-2-phenyl-ethylcarbamoyl]-2-(4-ethoxy-phenyl)-ethyl]-amide;

Cyclohexanecarboxylic acid [(R)-1-[(S)-1-(4-aminomethyl-benzylcarbamoyl)-2-phenyl-ethylcarbamoyl]-2-(4-ethoxy-phenyl)-ethyl]-amide;

Isoxazole-5-carboxylic acid [(R)-1-[(S)-1-(4-aminomethyl-benzylcarbamoyl)-2-phenyl-ethylcarbamoyl]-2-(4-ethoxy-phenyl)-ethyl]-amide;

Pyridine-2-carboxylic acid [(R)-1-[(S)-1-(4-aminomethyl-benzylcarbamoyl)-2-phenyl-ethylcarbamoyl]-2-(4-ethoxy-phenyl)-ethyl]-amide;

Benzo[b]thiophene-2-carboxylic acid[(R)-1-[(S)-1-(4-aminomethyl-benzylcarbamoyl)-2-phenyl-ethylcarbamoyl]-2-(4-ethoxy-phenyl)-ethyl]-amide;

(R)-N-[(S)-1-(4-Aminomethyl-benzylcarbamoyl)-2-phenyl-ethyl]-2-(4-chloro-benzenesulfonylamino)-3-(4-ethoxy-phenyl)-propionamide;

N-[(R)-1-[(S)-1-(4-Aminomethyl-benzylcarbamoyl)-2-phenyl-ethylcarbamoyl]-2-(4-ethoxy-phenyl)-ethyl]-3-chloro-benzamide;

N-[(R)-1-[(S)-1-(4-Aminomethyl-benzylcarbamoyl)-2-phenyl-ethylcarbamoyl]-2-(4-ethoxy-phenyl)-ethyl]-2-chloro-benzamide

N-[(R)-1-[(S)-1-(4-Aminomethyl-benzylcarbamoyl)-2-phenyl-ethylcarbamoyl]-2-(4-ethoxy-phenyl)-ethyl]-3-trifluoromethyl-benzamide;

N-[(R)-1-[(S)-1-(4-Aminomethyl-benzylcarbamoyl)-2-phenyl-ethylcarbamoyl]-2-(4-ethoxy-phenyl)-ethyl]-4-methyl-benzamide;

N-[(R)-1-[(S)-1-(4-Aminomethyl-benzylcarbamoyl)-2-phenyl-ethylcarbamoyl]-2-(4-ethoxy-phenyl)-ethyl]-3,4-dichloro-benzamide;

N-[(R)-1-[(S)-1-(4-Aminomethyl-benzylcarbamoyl)-2-phenyl-ethylcarbamoyl]-2-(4-ethoxy-phenyl)-ethyl]-4-methoxy-benzamide;

(S)-N-(4-Aminomethyl-benzyl)-2-[(R)-3-(4-ethoxy-phenyl)-2-(2-phenylacetylamino-acetylamino)-propionylamino]-3-phenyl-propionamide;

N-[(R)-1-[(S)-1-(4-Aminomethyl-benzylcarbamoyl)-2-phenyl-ethylcarbamoyl]-2-(4-ethoxy-phenyl)-ethyl]-4-fluoro-benzamide;

N-[(R)-1-[(S)-1-(4-Aminomethyl-benzylcarbamoyl)-2-phenyl-ethylcarbamoyl]-2-(4-ethoxy-phenyl)-ethyl]-6-methyl-nicotinamide;

N-[(R)-1-[(S)-1-(4-Aminomethyl-benzylcarbamoyl)-2-phenyl-ethylcarbamoyl]-2-(4-ethoxy-phenyl)-ethyl]-2-methyl-nicotinamide;

N-[(R)-1-[(S)-1-(4-Aminomethyl-benzylcarbamoyl)-2-phenyl-ethylcarbamoyl]-2-(4-ethoxy-phenyl)-ethyl]-2,6-dichloro-nicotinamide;

N-[(R)-1-[(S)-1-(4-Aminomethyl-benzylcarbamoyl)-2-phenyl-ethylcarbamoyl]-2-(4-ethoxy-phenyl)-ethyl]-5,6-dichloro-nicotinamide;

N-[(R)-1-[(S)-1-(4-Aminomethyl-benzylcarbamoyl)-2-phenyl-ethylcarbamoyl]-2-(4-ethoxy-phenyl)-ethyl]-2,3,6-trifluoro-isonicotinamide;

N-[(R)-1-[(S)-1-(4-Aminomethyl-benzylcarbamoyl)-2-phenyl-ethylcarbamoyl]-2-(4-ethoxy-phenyl)-ethyl]-3,3,3-trifluoro-propionamide;

2,4-Dimethyl-thiazole-5-carboxylic acid [(R)-1-[(S)-1-(4-aminomethyl-benzylcarbamoyl)-2-phenyl-ethylcarbamoyl]-2-(4-ethoxy-phenyl)-ethyl]-amide;

2-Methyl-thiazole-5-carboxylic acid [(R)-1-[(S)-1-(4-aminomethyl-benzylcarbamoyl)-2-phenyl-ethylcarbamoyl]-2-(4-ethoxy-phenyl)-ethyl]-amide;

3-Chloro-thiophene-2-carboxylic acid [(R)-1-[(S)-1-(4-aminomethyl-benzylcarbamoyl)-2-phenyl-ethylcarbamoyl]-2-(4-ethoxy-phenyl)-ethyl]-amide;

4-Methyl-thiazole-5-carboxylic acid[(R)-1-[(S)-1-(4-aminomethyl-benzylcarbamoyl)-2-phenyl-ethylcarbamoyl]-2-(4-ethoxy-phenyl)-ethyl]-amide;

Furan-2-carboxylic acid [(R)-1-[(S)-1-(4-aminomethyl-benzylcarbamoyl)-2-phenyl-ethylcarbamoyl]-2-(4-ethoxy-phenyl)-ethyl]-amide;

3-Methyl-thiophene-2-carboxylic acid [(R)-1-[(S)-1-(4-aminomethyl-benzylcarbamoyl)-2-phenyl-ethylcarbamoyl]-2-(4-ethoxy-phenyl)-ethyl]-amide;

N-[(R)-1-[(S)-1-(4-Aminomethyl-benzylcarbamoyl)-2-phenyl-ethylcarbamoyl]-2-(4-ethoxy-phenyl)-ethyl]-2-methoxy-isonicotinamide;

3-Methyl-1H-pyrrole-2-carboxylic acid [(R)-1-[(S)-1-(4-aminomethyl-benzylcarbamoyl)-2-phenyl-ethylcarbamoyl]-2-(4-ethoxy-phenyl)-ethyl]-amide;

3-Amino-thiophene-2-carboxylic acid [(R)-1-[(S)-1-(4-aminomethyl-benzylcarbamoyl)-2-phenyl-ethylcarbamoyl]-2-(4-ethoxy-phenyl)-ethyl]-amide;

N-[(R)-1-[(S)-1-(4-Aminomethyl-benzylcarbamoyl)-2-phenyl-ethylcarbamoyl]-2-(4-propoxy-phenyl)-ethyl]-benzamide;

N-[(R)-1-[(S)-1-(4-Aminomethyl-benzylcarbamoyl)-2-pyridin-2-yl-ethylcarbamoyl]-2-(4-ethoxy-phenyl)-ethyl]-benzamide;

N-[(R)-1-[(S)-1-(4-Aminomethyl-benzylcarbamoyl)-2-(3,4-dichloro-phenyl)-ethylcarb amoyl]-2-(4-ethoxy-phenyl)-ethyl]-benzamide;

N-[(R)-1-[(S)-1-(4-Aminomethyl-benzylcarbamoyl)-2-(4-chloro-phenyl)-ethylcarbamoyl]-2-(4-ethoxy-phenyl)-ethyl]-benzamide;

N-[(R)-1-[(S)-1-(4-Aminomethyl-benzylcarbamoyl)-2-(4-fluoro-phenyl)-ethylcarbamoyl]-2-(4-ethoxy-phenyl)-ethyl]-benzamide;

N-[(R)-1-[(S)-1-(4-Aminomethyl-benzylcarbamoyl)-2-pyridin-3-yl-ethylcarbamoyl]-2-(4-ethoxy-phenyl)-ethyl]-benzamide;

N-[(R)-1-[(S)-1-(4-Aminomethyl-benzylcarbamoyl)-2-(4-methoxy-phenyl)-ethylcarbamoyl]-2-(4-ethoxy-phenyl)-ethyl]-benzamide;

N-[(R)-1-[(S)-1-(4-Aminomethyl-benzylcarbamoyl)-2-pyridin-4-yl-ethylcarbamoyl]-2-(4-ethoxy-phenyl)-ethyl]-benzamide;

N-[(R)-1-[(S)-1-(4-Aminomethyl-benzylcarbamoyl)-2-(3-fluoro-phenyl)-ethylcarbamoyl]-2-(4-ethoxy-phenyl)-ethyl]-benzamide;

N-[(R)-1-[(S)-1-(4-Aminomethyl-benzylcarbamoyl)-2-thiophen-2-yl-ethylcarbamoyl]-2-(4-ethoxy-phenyl)-ethyl]-benzamide;

N-[(R)-1-[(S)-1-(4-Aminomethyl-benzylcarbamoyl)-2-thiophen-3-yl-ethylcarbamoyl-]2-(4-ethoxy-phenyl)-ethyl]-benzamide;

N-[(R)-1-[(S)-1-(4-Aminomethyl-benzylcarbamoyl)-2-thiazol-4-yl-ethylcarbamoyl]-2-(4-ethoxy-phenyl)-ethyl]-benzamide;

N-[(R)-1-[(S)-1-(4-Aminomethyl-benzylcarbamoyl)-2-benzo[b]thiophen-3-yl-ethylcarbamoyl]-2-(4-ethoxy-phenyl)-ethyl]-benzamide;

N-[(R)-1-[(S)-1-(4-Aminomethyl-3-fluoro-benzylcarbamoyl)-2-phenyl-ethylcarbamoyl]-2-(4-ethoxy-phenyl)-ethyl]-benzamide;

N-[(R)-1-[(S)-1-(4-Aminomethyl-3-chloro-benzylcarbamoyl)-2-phenyl-ethylcarbamoyl]-2-(4-ethoxy-phenyl)-ethyl]-benzamide;

Pyridine-2-carboxylic acid [(R)-1-[(S)-1-(4-aminomethyl-benzylcarbamoyl)-2-thiophen-2-yl-ethylcarbamoyl]-2-(4-ethoxy-phenyl)-ethyl]-amide;

N-[(R)-1-[(S)-1-(4-Aminomethyl-benzylcarbamoyl)-2-pyridin-2-yl-ethylcarbamoyl]-2-(4-ethoxy-phenyl)-ethyl]-4-methoxy-benzamide;

Pyridine-2-carboxylic acid [(R)-1-[(S)-1-(4-aminomethyl-3-chloro-benzylcarbamoyl)-2-phenyl-ethylcarbamoyl]-2-(4-ethoxy-phenyl)-ethyl]-amide;

N-[(R)-1-[(S)-1-(4-Aminomethyl-benzylcarbamoyl)-2-pyridin-3-yl-ethylcarbamoyl]-2-(4-ethoxy-phenyl)-ethyl]-4-methoxy-benzamide;

N-[(R)-1-[(S)-1-(4-Aminomethyl-benzylcarbamoyl)-2-(3,4-difluoro-phenyl)-ethylcarbamoyl]-2-(4-ethoxy-phenyl)-ethyl]-isonicotinamide;

Thiophene-2-carboxylic acid [(R)-1-[(S)-1-(4-aminomethyl-benzylcarbamoyl)-2-pyridin-3-yl-ethylcarbamoyl]-2-(4-ethoxy-phenyl)-ethyl]-amide;

N-[(R)-1-[(S)-1-(4-Aminomethyl-benzylcarbamoyl)-2-pyridin-2-yl-ethylcarbamoyl]-2-(4-ethoxy-phenyl)-ethyl]-4-chloro-benzamide;

N-[(R)-1-[(S)-1-(4-Aminomethyl-benzylcarbamoyl)-2-pyridin-2-yl-ethylcarbamoyl]-2-(4-ethoxy-phenyl)-ethyl]-4-methyl-benzamide;

Pyridine-2-carboxylic acid [(R)-1-[(S)-1-(4-aminomethyl-benzylcarbamoyl)-2-(3,4-dichloro-phenyl)-ethylcarbamoyl]-2-(4-ethoxy-phenyl)-ethyl]-amide;

(R)-N-[(S)-1-(4-Aminomethyl-benzylcarbamoyl)-2-pyridin-2-yl-ethyl]-3-(4-ethoxy-phenyl)-2-propionylamino-propionamide;

N-[(R)-1-[(S)-1-(4-Aminomethyl-3-fluoro-benzylcarbamoyl)-2-phenyl-ethylcarbamoyl]-2-(4-ethoxy-phenyl)-ethyl]-isonicotinamide;

Pyridine-2-carboxylic acid [(R)-1-[(S)-1-(4-aminomethyl-3-fluoro-benzylcarbamoyl)-2-phenyl-ethylcarbamoyl]-2-(4-ethoxy-phenyl)-ethyl]-amide;

Thiophene-2-carboxylic acid [(R)-1-[(S)-1-(4-aminomethyl-benzylcarbamoyl)-2-(3,4-dichloro-phenyl)-ethylcarbamoyl]-2-(4-ethoxy-phenyl)-ethyl]-amide;

(R)-N-[(S)-1-(4-Aminomethyl-benzylcarbamoyl)-2-pyridin-3-yl-ethyl]-3-(4-ethoxy-phenyl)-2-propionylamino-propionamide;

N-[(R)-1-[(S)-1-(4-Aminomethyl-benzylcarbamoyl)-2-(3,4-dichloro-phenyl)-ethylcarbamoyl]-2-(4-ethoxy-phenyl)-ethyl]-isonicotinamide;

N-[(R)-1-[(S)-1-(4-Aminomethyl-benzylcarbamoyl)-2-(3,4-dichloro-phenyl)-ethylcarbamoyl]-2-(4-ethoxy-phenyl)-ethyl]-3,3,3-trifluoro-propionamide;

N-[(R)-1-[(S)-1-(4-Aminomethyl-benzylcarbamoyl)-2-pyridin-3-yl-ethylcarbamoyl]-2-(4-ethoxy-phenyl)-ethyl]-4-chloro-benzamide;

Isoxazole-5-carboxylic acid [(R)-1-[(S)-1-(4-aminomethyl-benzylcarbamoyl)-2-pyridin-3-yl-ethylcarbamoyl]-2-(4-ethoxy-phenyl)-ethyl]-amide;

N-[(R)-1-[(S)-1-(4-Aminomethyl-benzylcarbamoyl)-2-pyridin-3-yl-ethylcarbamoyl]-2-(4-ethoxy-phenyl)-ethyl]-4-methyl-benzamide;

N-[(R)-1-[(S)-1-(4-Aminomethyl-benzylcarbamoyl)-2-(3,4-difluoro-phenyl)-ethylcarbamoyl]-2-(4-ethoxy-phenyl)-ethyl]-benzamide;

3-Chloro-thiophene-2-carboxylic acid [(R)-1-[(S)-1-(4-aminomethyl-benzylcarbamoyl)-2-pyridin-3-yl-ethylcarbamoyl]-2-(4-ethoxy-phenyl)-ethyl]-amide;

N-[(R)-1-[(S)-1-(4-Aminomethyl-benzylcarbamoyl)-2-(1H-indo1-3-yl)-ethylcarb amoyl]-2-(4-ethoxy-phenyl)-ethyl]-benzamide;

N-[(R)-1-[(S)-1-(4-Aminomethyl-benzylcarbamoyl)-2-benzo [b]thiophen-3-yl-ethylcarb amoyl]-2-(4-ethoxy-phenyl)-ethyl]-is onicotinamide;

3-Acetylamino-thiophene-2-carboxylic acid-[(R)-1-[(S)-1-(4-aminomethyl-benzylcarbamoyl)-2-phenyl-ethyl carb amoyl]-2-(4-ethoxy-phenyl)-ethyl]-amide;

N-[(R)-1-[(S)-1-(4-Aminomethyl-benzylcarbamoyl)-2-(2-fluoro-phenyl)-ethylcarb amoyl]-2-(4-ethoxy-phenyl)-ethyl]-benzamide;

3-Methyl-thiophene-2-carboxylic acid [(R)-1-[(S)-1-(4-aminomethyl-benzylcarbamoyl)-2-pyridin-3-yl-ethylcarb amoyl]-2-(4-ethoxy-phenyl)-ethyl]-amide;

N-[(R)-1-[(S)-1-(4-Aminomethyl-3-methyl-benzylcarbamoyl)-2-phenyl-ethylcarb amoyl]-2-(4-ethoxy-phenyl)-ethyl]-benzamide;

3-Amino-thiophene-2-carboxylic acid [(R)-1-[(S)-1-(4-aminomethyl-benzylcarb amoyl)-2-thi azol-4-yl-ethylcarb amoyl]-2-(4-ethoxy-phenyl)-ethyl]-amide;

3-Chloro-thiophene-2-carboxylic acid [(R)-1-[(S)-1-(4-aminomethyl-benzylcarbamoyl)-2-thi azol-4-yl-ethylcarb amoyl]-2-(4-ethoxy-phenyl)-ethyl]-amide;

N-[(R)-1-[(S)-1-(4-Aminomethyl-benzylcarbamoyl)-2-thi azol-4-yl-ethylcarbamoyl]-2-(4-ethoxy-phenyl)-ethyl]-4-methyl-benzamide;

3-Methyl-1H-pyrrole-2-carboxylic acid [(R)-1-[(S)-1-(4-aminomethyl-benzylcarbamoyl)-2-benzo [b]thiophen-3-yl-ethylcarb amoyl]-2-(4-ethoxy-phenyl)-ethyl]- amide;

3-Amino-thiophene-2-carboxylic acid [(R)-1-[(S)-1-(4-aminomethyl-benzylcarbamoyl)-2-thi azol-4-yl-ethylcarb amoyl]-2-(4-ethoxy-phenyl)-ethyl]-amide;

3-Acetylamino-thiophene-2-carboxylic acid [(R)-1-[(S)-1-(4-aminomethyl-benzylcarbamoyl)-2-benzo [b]thiophen-3-yl-ethylcarb amoyl]-2-(4-ethoxy-phenyl)-ethyl]- amide;

N-[(R)-1-[(S)-1-(4-Aminomethyl-benzylcarbamoyl)-2-pyridin-3-yl-ethylcarbamoyl]-2-(4-ethoxy-phenyl)-ethyl]-3-methyl-benzamide;

N-[(R)-1-[(S)-1-(4-Aminomethyl-benzylcarbamoyl)-2-pyridin-3-yl-ethylcarbamoyl]-2-(4-ethoxy-phenyl)-ethyl]-2-methyl-benzamide;

3,5-Dimethyl-1H-pyrrole-2-carboxylic acid [(R)-1-[(S)-1-(4-aminomethyl-benzylcarbamoyl)-2-phenyl-ethylcarbamoyl]-2-(4-ethoxy-phenyl)-ethyl]-amide;

N-[(R)-1-[(S)-1-(4-Aminomethyl-3-methyl-benzylcarbamoyl)-2-pyridin-3-yl-ethylcarbamoyl]-2-(4-ethoxy-phenyl)-ethyl]-benzamide;

3-Acetylamino-thiophene-2-carboxylic acid [(R)-1-[(S)-1-(4-aminomethyl-benzylcarbamoyl)-2-thiophen-3-yl-ethylcarbamoyl]-2-(4-ethoxy-phenyl)-ethyl]-amide;

3-Amino-thiophene-2-carboxylic acid [(R)-1-[(S)-1-(4-aminomethyl-benzylcarbamoyl)-2-benzo[b]thiophen-3-yl-ethylcarbamoyl]-2-(4-ethoxy-phenyl)-ethyl]-amide;

3-Acetylamino-thiophene-2-carboxylic acid [(R)-1-[(S)-1-(4-aminomethyl-benzylcarbamoyl)-2-benzo[b]thiophen-3-yl-ethylcarbamoyl]-2-(4-ethoxy-phenyl)-ethyl]-amide;

3-Chloro-thiophene-2-carboxylic acid [(R)-1-{[(S)-1-(4-aminomethyl-benzylcarbamoyl)-2-phenyl-ethyl]-methyl-carbamoyl}-2-(4-ethoxy-phenyl)-ethyl]-amide;

N-[(R)-1-[(1S,2R)-1-(4-Aminomethyl-benzylcarbamoyl)-2-hydroxy-2-phenyl-ethylcarbamoyl]-2-(4-ethoxy-phenyl)-ethyl]-benzamide;

3-Chloro-thiophene-2-carboxylic acid [(R)-1-[(1S,2R)-1-(4-aminomethyl-benzylcarbamoyl)-2-hydroxy-2-phenyl-ethylcarbamoyl]-2-(4-ethoxy-phenyl)-ethyl]- amide; and

N-{(R,S)-1-[(S)-1-(4-Aminomethyl-benzylcarbamoyl)-2-phenyl-ethylcarbamoyl]-2-[4-(2,2,2-trifluoro-ethoxy)-phenyl]-ethyl}-benzamide;

and pharmaceutically acceptable salts thereof.

In particularly preferred embodiments, the active ingredient is N-[(R)-1-[(S)-1-(4-aminomethyl-benzylcarbamoyl)-2-phenyl-ethylcarbamoyl]-2-(4-ethoxy-phenyl)-ethyl]-benzamide, or a pharmaceutically acceptable salt thereof. N-[(R)-1-[(S)-1-(4-aminomethyl-benzylcarbamoyl)-2-phenyl-ethylcarbamoyl]-2-(4-ethoxy-phenyl)-ethyl]-benzamide is a plasma kallikrein inhibitor. In a more preferred embodiment, the active ingredient is N-[(R)-1-[(S)-1-(4-aminomethyl-benzylcarbamoyl)-2-phenyl-ethylcarbamoyl]-2-(4-ethoxy-phenyl)-ethyl]-benzamide hydrochloride.

The compounds used in the invention can be prepared according to known procedures, especially those described by Evans et al. (“Benzylamine derivatives as inhibitors of plasma kallikrein” WO2013/005045), using appropriate materials. Moreover, by utilising these procedures, one of ordinary skill in the art can readily prepare additional compounds that can be used in the compositions of the invention.

The compounds used in the invention may be isolated in the form of their pharmaceutically acceptable salts, such as those described herein. The pharmaceutically acceptable salt is typically a hydrochloride salt.

Excipients

The compositions of the present invention are adapted for parenteral administration. In particular, the compositions of the present invention may be adapted for injection into the eye. More particularly, the compositions of the present invention may be adapted for intravitreal injection.

The compositions of the invention include one or more pharmaceutically acceptable excipients. The term ‘excipient’ is used herein to describe any ingredient other than the active ingredient which may impart either a functional (e.g. injectability, stability enhancing, drug release rate controlling) and/or a non-functional (e.g. processing aid or diluent) characteristic to the formulations. The choice of excipient will to a large extent depend on factors such as the particular mode of administration, the effect of the excipient on solubility and stability, and the nature of the dosage form.

The compositions of the invention include at least one buffer. The use of a buffer can minimize fluctuations in pH, which may improve stability and/or improve the tolerability of the composition in a subject upon administration. Suitable buffers that can be used in the compositions of the invention include histidine, acetate, citrate, cacodylate, bis-tris, maleate, piperazine, MES (2-(N-morpholino)ethanesulfonic acid), tartrate, lactate; succinate; sulfate; phosphate; alanine; imidazole; arginine and asparagine. Typically, the buffer is selected from histidine, maleate and citrate. Preferably, the buffer is histidine. The pH of the buffer will typically be between about 2 and about 10, e.g. about pH 2, 3, 4, 5, 6, 7, 8, 9 or 10. Typically, the pH of the buffer will be between about 4 and about 8. Preferably, the pH of the buffer will be between about 5 and about 7.5. More preferably, the pH of the buffer will be at a pH of between about 5 and about 6. In an embodiment, the buffer is not PBS (phosphate buffered saline).

The pH of the buffer may be adjusted by the addition of an acid or a base. For example, the pH of the buffer may be adjusted with hydrochloric acid. The buffers referred to in this invention are also intended to include salts of the buffer. For example, histidine buffer includes histidine hydrochloride buffer.

The compositions of the invention comprise a buffer in an amount from about 0.0001% to about 1%, or from about 0.001% to about 0.32%, optionally from about 0.01% to about 0.16%. Preferably, the compositions of the invention comprise a buffer in an amount from about 0.01% to about 0.08% by weight of the composition. Typically, the compositions of the invention comprise a buffer in an amount of about 0.01%, 0.02%, 0.03% or 0.04% by weight of the composition.

The compositions of the invention include at least one non-ionic tonicity agent. The use of a non-ionic tonicity agent allows control of the osmolality of the composition. The non-ionic tonicity agent is typically a carbohydrate and is preferably a sugar. The non-ionic tonicity agent may be selected from the group comprising glycerine; sugars, e.g. glucose, mannitol, sorbitol, trehalose, dextrose, lactose, maltose, fructose, sucrose, and inositol; hydroxyethyl starch, e.g. hetastarch and pentastarch. The non-ionic tonicity agent is typically dextrose or trehalose. Preferably, the non-ionic tonicity agent is trehalose.

The compositions of the invention may be free, or substantially free, of saline. The compositions of the invention are preferably free of phosphate buffered saline.

The compositions of the invention may contain histidine as the buffer and dextrose or trehalose as the non-ionic tonicity agent and may optionally be free, or substantially free, of phosphate buffered saline.

The compositions of the invention may be hypotonic, isotonic or hypertonic. It may be desirable that a composition for intravitreal injection is isotonic to the vitreous, i.e. has the same effective osmolality as the vitreous, so as not to disrupt the fluid balance of the vitreous and surrounding tissues.

The compositions of the invention comprise a non-ionic tonicity agent in an amount from about 0.1% to about 30% by weight of the composition, e.g. about 0.1%, 0.2%, 0.3%, 0.4%, 0.5%, 0.6%, 0.7%, 0.8%, 0.9%, 1%, 2.5%, 5%, 10%, 15%, 20%, 25% or 30% by weight of the composition. Typically, the compositions of the invention comprise a non-ionic tonicity agent in an amount from about 1% to about 20%, or from about 5% to about 15%, or from about 7% to about 12% by weight of the composition, or from about 8% to about 10% by weight of the composition. Typically, the compositions of the invention comprise a non-ionic tonicity agent in an amount of about 8%, 9% or 10% by weight of the composition.

The compositions of the invention typically have an osmolality of from about 250 to about 350 mOsmol/kg. For example, the compositions may have an osmolality of 250, 260, 270, 280, 290, 300, 310, 320, 330, 340 or 350 mOsmol/kg. The skilled person will understand that the amount of non-ionic tonicity agent used may vary depending on the particular choice of agent and on the other components in the composition.

The composition may include a non-ionic surfactant, such as carboxylic esters, polyethylene glycol esters, glycol esters of fatty acids, ethoxylated aliphatic alcohols, polyoxyethelene surfactants, sorbitol esters, ethoxylated derivatives of sorbitol esters, glycol esters of fatty acids, and poloxamers. Polyoxyethelene surfactants include polyoxyethylenesorbitan fatty acid esters, which are also referred to as polysorbates, e.g. polysorbate 80 (polyoxyethylene sorbitan monooleate, Tween® 80), polysorbate 40 (polyoxyethylene sorbitan monopalmitate, Tween® 40) and polysorbate 20 (polyoxyethylene sorbitan monolaurate, Tween® 20). Preferably, the non-ionic surfactant is a polyoxyethylenesorbitan fatty acid ester. More preferably, the non-ionic surfactant is polysorbate 20.

Alternatively, the compositions of the invention may be free, or substantially free, of non-ionic surfactants, such as carboxylic esters, polyethylene glycol esters, glycol esters of fatty acids, ethoxylated aliphatic alcohols, polyoxyethelene surfactants, sorbitol esters, ethoxylated derivatives of sorbitol esters, glycol esters of fatty acids, and poloxamers. Polyoxyethelene surfactants include polyoxyethylenesorbitan fatty acid esters, which are also referred to as polysorbates, e.g. polysorbate 80 (polyoxyethylene sorbitan monooleate, Tween® 80), polysorbate 40 (polyoxyethylene sorbitan monopalmitate, Tween® 40) and polysorbate 20 (polyoxyethylene sorbitan monolaurate, Tween® 20). The compositions of the invention are preferably free of polysorbate, e.g. polysorbate 20.

The compositions of the invention may contain histidine as the buffer and dextrose or trehalose as the non-ionic tonicity agent and may optionally be free, or substantially free, of polysorbate, e.g. polysorbate 20.

The composition may include an antioxidant, such as acetone, sodium bisulfite, butylated hydroxy anisole, butylated hydroxy toluene, cysteine, cysteinate HCl, dithionite sodium, gentisic acid, gentisic acid ethanolamine, glutamate monosodium, formaldehyde sulfoxylate sodium, metabisulfite potassium, metabisulfite sodium, monothioglycerol, propyl gallate, sulfite sodium, thioglycolate sodium or ascorbic acid. Alternatively, in particular for intraocular use of the composition, packaging may be configured in a manner that controls the potential for oxidation of the composition, including for example purging with an inert gas during manufacture.

In the compositions of the invention the active ingredient may have a half-life, t_{1/2}, in vitreous humor of at least about 1 day, e.g. at least about 3 days, at least about 5 days, or about 7 days or more. In the compositions of the invention the active ingredient may have a t_{1/2} in vitreous humor of less than about 40 days, e.g. less than about 30 days, less than about 20 days. In the compositions of the invention the active ingredient may have a t_{1/2} in vitreous humor of between about 1 day and about 40 days, e.g. between about 3 days and about 30 days, or about 5 days and about 20 days.

In the compositions of the invention the active ingredient may have a half-life, t_{1/2}, in the retina of at least about 1 day, e.g. at least about 3 days, at least about 5 days, or about 7 days or more. In the compositions of the invention the active ingredient may have a tᵥ₂ in the retina of less than about 40 days, e.g. less than about 30 days, less than about 20 days. In the compositions of the invention the active ingredient may have a t_{i/2} in the retina of between about 1 day and about 40 days, e.g. between about 3 days and about 30 days, or about 5 days and about 20 days.

In the compositions of the invention the active ingredient may have a half-life, t_{1/2}, in the retina-choroid complex of at least about 1 day, e.g. at least about 3 days, at least about 5 days, or about 7 days or more. In the compositions of the invention the active ingredient may have a t_{1/2} in the retina-choroid complex of less than about 40 days, e.g. less than about 30 days, less than about 20 days. In the compositions of the invention the active ingredient may have a t_{1/2} in the retina-choroid complex of between about 1 day and about 40 days, e.g. between about 3 days and about 30 days, or about 5 days and about 20 days.

Additional Therapeutic Agents

The compositions of the invention may include one or more other therapeutic agents. For example, the compositions may include one or more of an agent that inhibits platelet-derived growth factor (PDGF), an agent that inhibits endothelial growth factor (VEGF), and an agent that inhibits integrins, for example integrin alpha5beta1. The compositions may also include one or more steroids. The compositions may also include other agents that inhibit plasma kallikrein and/or other inhibitors of inflammation. The composition may also include antagonists of bradykinin, for example antagonists of the bradykinin B2 receptor such as the drug icatibant.

Specific examples of therapeutic agents that may be included in the compositions of the invention include those disclosed in EP2281885A and by S. Patel in Retina, 2009 June; 29(6 Suppl):545-8.

In some embodiments, the plasma kallikrein inhibitor of formula I and the one or more other therapeutic agents may exist in the same aqueous solution pharmaceutical composition. In other embodiments, the plasma kallikrein inhibitor of formula I and the one or more other therapeutic agents may exist in different pharmaceutical compositions (one of which is an aqueous solution pharmaceutical composition). The compositions may be administered separately, sequentially or simultaneously.

Administration, Medical Treatments and Uses

The invention also provides a method for treating a disease or condition mediated by plasma kallikrein comprising parenteral administration of a pharmaceutical composition of the invention to a mammal.

The invention also provides a pharmaceutical composition of the invention for use in treating a disease or condition mediated by plasma kallikrein.

The invention also provides the use of a pharmaceutical composition of the invention in the manufacture of a medicament for the treatment of a disease or condition mediated by plasma kallikrein, wherein said treatment comprises the administration of a pharmaceutical composition of the invention.

The uses and methods are useful for the treatment of a disease or condition mediated by plasma kallikrein. For example, the uses and methods are useful for the treatment of impaired visual acuity, diabetic retinopathy, retinal vascular permeability associated with diabetic retinopathy, diabetic macular edema, retinal vascular occlusion, hereditary angioedema, diabetes, pancreatitis, cerebral haemorrhage, cerebral haemorrhage in hyperglycemic patients, nephropathy, cardiomyopathy, neuropathy, inflammatory bowel disease, arthritis, inflammation, septic shock, hypotension, cancer, adult respiratory distress syndrome, disseminated intravascular coagulation, blood coagulation during cardiopulmonary bypass surgery and bleeding from post-operative surgery. Preferably, the uses and methods are useful for the treatment of impaired visual acuity, diabetic retinopathy, retinal vascular permeability associated with diabetic retinopathy, diabetic macular edema, and retinal vascular occlusion. More preferably, the uses and methods are useful for the treatment of retinal vascular permeability associated with diabetic retinopathy or diabetic macular edema. In some embodiments, the uses and methods are useful for the treatment of microvascular complications of a disease state.

The compositions of the invention are suitable for parenteral administration. Accordingly, the compounds of the invention may be administered directly e.g. into the blood stream, into subcutaneous tissue, into muscle, into the eye or into an internal organ. Suitable means for parenteral administration include intraarterial, intraperitoneal, intrathecal, intraventricular, intraurethral, intrasternal, intracranial, intramuscular, intrasynovial, subcutaneous, intravenous, intravitreal, intravitreal injection or intravitreous. In some embodiments, the compositions may be administered via surgical incision into the subcutaneous tissue, muscular tissue or directly into specific organs.

In one preferred embodiment, the composition is administered by intravitreal injection. To improve patient compliance when administering the compositions by intravitreal injection, it is preferred to administer the composition relatively infrequently. For example, the composition may be administered from about once every two weeks to about once every 6 months, e.g. from about once every month to about once every 6 months, or about once a month, or about once every two months, three months, four months, or five months. Preferably, the composition will be administered once every one month or once every two months or once every three months. Optionally, the composition will be administered once every one month.

Suitable devices for parenteral administration include needle (including microneedle) injectors, needle-free injectors and infusion techniques.

In some embodiments, the uses and methods involve combination therapy. For example, the uses and methods may further comprise laser treatment of the retina. The combination of laser therapy with intravitreal injection of an inhibitor of VEGF for the treatment of diabetic macular edema is known (Elman M, Aiello L, Beck R, et al. “Randomized trial evaluating ranibizumab plus prompt or deferred laser or triamcinolone plus prompt laser for diabetic macular edema”. Ophthalmology. 27 Apr. 2010).

It is envisaged that the compositions of the invention will take the form of sterile aqueous solutions. The preparation of parenteral formulations under sterile conditions, for example, by lyophilisation and reconstitution, may readily be accomplished using standard pharmaceutical techniques well known to those skilled in the art. For example, a suitable method for sterilising the compositions of the present invention may be terminal sterilisation, or sterile filtration followed by aseptic fill-finish. The terminal sterilisation method, sterile filtration and aseptic processing are described in US Pharmacopeia USP<1211> Sterilization and Sterility Assurance of Compendial Articles and terminal sterilisation is further described in US Pharmacopeia USP<1222> Terminally Sterilized Pharmaceutical Products-Parametric Release. (See United States Pharmacopeia (USP) 37, NF 32).

The compositions may be administered to the patient under the supervision of an attending physician.

Processes

The present invention further relates to processes for preparing pharmaceutical compositions of the invention.

In one embodiment, a method for preparing the pharmaceutical composition of the invention is provided, comprising the steps of
(a) preparing a solution of at least one non-ionic tonicity agent and at least one buffer in water;
(b) dissolving a compound of formula I, or a pharmaceutically acceptable salt thereof, in the solution prepared in step (a);
wherein the at least one non-ionic tonicity agent, the at least one buffer, and the compound of formula I are as defined herein.

Preferably, the water used in step (a) is sterile water for injection.

The method may further comprise the step of:
(c) adding an aqueous solution of at least one non-ionic tonicity agent and at least one buffer to the solution prepared in step (b); and/or
(d) sterilising the solution.

Preferably, the sterilisation in step (d) is performed by sterile filtration.

The present invention also provides a method for preparing the pharmaceutical composition of the invention, comprising adding water to a non-aqueous formulation comprising at least one non-ionic tonicity agent, at least one buffer and an active ingredient, wherein said active ingredient is a compound of formula I or a pharmaceutically acceptable salt thereof, and wherein the at least one non-ionic tonicity agent, the at least one buffer, and the compound of formula I are as defined herein.

Definitions

The term “aqueous” means that the composition includes water as a solvent. Typically, the content of water in the composition is greater than or equal to about 35% by weight, preferably more than about 50% by weight of the composition, e.g. more than about 60%, 65%, 70%, 75%, 80%, 85%, 90%, 95%, 97%, 98% or 99% by weight of the composition.

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x±10%.

The term “alkyl” includes saturated hydrocarbon residues including:
linear groups up to 10 carbon atoms (C₁-C₁₀), or of up to 6 carbon atoms (C₁-C₆), or of up to 4 carbon atoms (C₁-C₄). Examples of such alkyl groups include, but are not limited, to C₁-methyl, C₂-ethyl, C₃-propyl and C₄-n-butyl;
branched groups of between 3 and 10 carbon atoms (C₃-C₁₀), or of up to 7 carbon atoms (C₃-C₇), or of up to 4 carbon atoms (C₃-C₄). Examples of such alkyl groups include, but are not limited to, C₃-iso-propyl, C₄-sec-butyl, C₄-iso-butyl, C₄-tert-butyl and C₅-neo-pentyl;

each optionally substituted as stated above.

The term “alkoxy” includes O-linked hydrocarbon residues including:
linear groups of between 1 and 6 carbon atoms (C₁-C₆), or of between 1 and 4 carbon atoms (C₁-C₄). Examples of such alkoxy groups include, but are not limited to, C₁-methoxy, C₂-ethoxy, C₃-n-propoxy and C₄-n-butoxy;
branched groups of between 3 and 6 carbon atoms (C₃-C₆) or of between 3 and 4 carbon atoms (C₃-C₄). Examples of such alkoxy groups include, but are not limited to, C₃-iso-propoxy, and C₄-sec-butoxy and tert-butoxy;

each optionally substituted as stated above.

Unless otherwise stated, halo is selected from Cl, F, Br and I.

Cycloalkyl is as defined above. Cycloalkyl groups may contain from 3 to 10 carbon atoms, or from 4 to 10 carbon atoms, or from 5 to 10 carbon atoms, or from 4 to 6 carbon atoms. Examples of suitable monocyclic cycloalkyl groups include cyclopropyl, cyclobutyl, cyclopentyl, cyclohexyl and cycloheptyl. Examples of suitable bicyclic cycloalkyl groups include decahydronaphthalene and octahydro-1H-indene Examples of suitable cycloalkyl groups, when fused with aryl, include indanyl and 1,2,3,4-tetrahydronaphthyl.

Heterocycloalkyl is as defined above. Examples of suitable heterocycloalkyl groups include oxiranyl, aziridinyl, azetidinyl, tetrahydrofuranyl, pyrrolidinyl, tetrahydropyranyl, piperidinyl, N-methylpiperidinyl, morpholinyl, N-methyl morpholinyl, piperazinyl, N-methylpiperazinyl, azepanyl, oxazepanyl and diazepanyl.

Aryl is as defined above. Typically, aryl will be optionally substituted with 1, 2 or 3 substituents. Optional substituents are selected from those stated above. Examples of suitable aryl groups include phenyl and naphthyl (each optionally substituted as stated above).

Heteroaryl is as defined above. Examples of suitable heteroaryl groups include thienyl, furanyl, pyrrolyl, pyrazolyl, imidazoyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl, indolyl, benzimidazolyl, benzotriazolyl, quinolinyl and isoquinolinyl (optionally substituted as stated above).

The term “C-linked”, such as in “C-linked heterocycloalkyl”, means that the heterocycloalkyl group is joined to the remainder of the molecule via a ring carbon atom.

The term “N-linked”, such as in “N-linked heterocycloalkyl”, means that the heterocycloalkyl group is joined to the remainder of the molecule via a ring nitrogen atom.

The term “O-linked”, such as in “O-linked hydrocarbon residue”, means that the hydrocarbon residue is joined to the remainder of the molecule via an oxygen atom.

In groups such as —COalkyl and —(CH₂)_{b}COOR¹⁰, “—” denotes the point of attachment of the substituent group to the remainder of the molecule.

“Pharmaceutically acceptable salt” means a physiologically or toxicologically tolerable salt and includes, when appropriate, pharmaceutically acceptable base addition salts and pharmaceutically acceptable acid addition salts. For example (i) where a compound contains one or more acidic groups, for example carboxy groups, pharmaceutically acceptable base addition salts that can be formed include sodium, potassium, calcium, magnesium and ammonium salts, or salts with organic amines, such as, diethylamine, N-methyl-glucamine, diethanolamine or amino acids (e.g. lysine) and the like; (ii) where a compound contains a basic group, such as an amino group, pharmaceutically acceptable acid addition salts that can be formed include hydrochlorides, hydrobromides, sulfates, phosphates, acetates, citrates, lactates, tartrates, mesylates, succinates, oxalates, phosphates, esylates, tosylates, benzenesulfonates, naphthalenedisulphonates, maleates, adipates, fumarates, hippurates, camphorates, xinafoates, p-acetamidobenzoates, dihydroxybenzoates, hydroxynaphthoates, succinates, ascorbates, oleates, bisulfates and the like.

Hemisalts of acids and bases can also be formed, for example, hemisulfate and hemicalcium salts.

For a review of suitable salts, see “Handbook of Pharmaceutical Salts: Properties, Selection and Use” by Stahl and Wermuth (Wiley-VCH, Weinheim, Germany, 2002).

“Prodrug” refers to a compound which is convertible in vivo by metabolic means (e.g. by hydrolysis, reduction or oxidation) to a compound of the invention. Suitable groups for forming prodrugs are described in ‘The Practice of Medicinal Chemistry, 2^{nd} Ed. pp561-585 (2003) and in F. J. Leinweber, Drug Metab. Res., 1987, 18, 379.

Where compounds used in the compositions of the invention exist in one or more geometrical, optical, enantiomeric, diastereomeric and tautomeric forms, including but not limited to cis-and trans-forms, E-and Z-forms, R-, S-and meso-forms, keto-, and enol-forms, then, unless otherwise stated, a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers can be prepared by the application or adaptation of known methods (e.g. asymmetric synthesis).

A reference to a particular compound also includes all isotopic variants.

In the context of the present invention, references herein to “treatment” include references to curative, palliative and prophylactic treatment.

### MODES FOR CARRYING OUT THE INVENTION

The invention is further illustrated by the followings examples. It will be appreciated that the examples are for illustrative purposes only and are not intended to limit the invention as described above. Modification of detail may be made without departing from the scope of the invention. In the following examples, the following abbreviations and definitions are used:

| | |
|---|---|
| aq | Aqueous solution |
| hrs | Hours |
| IPA | iso-propanol |
| Me | Methyl |
| MeCN | Acetonitrile |
| MeOH | Methanol |
| Min | Minutes |
| QS | Quantum satis (sufficient quantity) |
| Ph | Phenyl |
| RRT | Relative retention time |
| SWFI | Sterile water for injection |
| rt | room temperature |

Osmolality

Osmolality was determined using a calibrated osmometer in compliance with USP<785> (freezing point depression). (See United States Pharmacopeia (USP) 37, NF 32).

Particulate Matter

Particulate matter in the pharmaceutical compositions was measured using the microscopic particle count test described in USP <789> (Particulate matter in ophthalmic solutions) (See United States Pharmacopeia (USP) 37, NF 32).

Synthetic Examples

Compounds of formula I may be prepared according to the methods described in Evans et al. (“Benzylamine derivatives as inhibitors of plasma kallikrein” WO2013/005045). N-[(R)-1-[(S)-1-(4-Aminomethyl-benzylcarbamoyl)-2-phenyl-ethylcarbamoyl]-2-(4-ethoxy-phenyl)-ethyl]-benzamide hydrochloride (“Compound 1”) can be manufactured using methods disclosed in WO2014/006414.

The structure of Compound 1 is shown below:

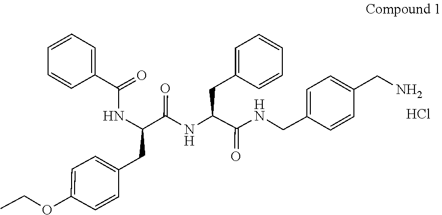 [SMILES: CCOc1ccc(C[C@@H](NC(=O)c2ccccc2)C(=O)N[C@@H](Cc2ccccc2)C(=O)NCc2ccc(CN)cc2)cc1 ; Cl]

Concentrations

Concentrations and dose levels defined in the examples below are based on the amount of free base of Compound 1.

Example 1:

Formulations of 10, 30, 100 μg/mL solution of Compound 1 in 0.01% polysorbate 20, 8.7% trehalose, 0.155% histidine (10 mM), QS SWFI were prepared and dosed at 0.5, 1.5, 5 μg/eye. Further details are provided below.

Vehicle Preparation

Hood and labware were sanitized with 70% IPA. Hydrochloric acid (5.00 mL) was made up to volume (50.0 mL) with sterile water for injection (SWFI). Polysorbate 20 (5.00 g) was made up to volume (50.0 mL) with SWFI.

Trehalose (43.53 g), histidine (0.78 g), previously prepared dilute hydrochloric acid (2.06 mL) and previously prepared polysorbate 20 solution (0.50 mL) were dissolved in SWFI and made up to volume (500.0 mL). The solution was vacuum filtered through sterile apparatus.

Dose Preparations

Compound 1 and the vehicle were removed from the refrigerator and allowed to come to rt.

150 mL of vehicle was sterile filtered as the first step in the dose formulation preparation.

Three separate 5mg samples of Compound 1 were weighed out and placed in three separate sterile containers. A volume (approximately 20 mL) of vehicle was added to each sterile container, wherein the volume (in mL) is equivalent to approximately 4× the mass (in mg) of free base Compound 1 per sterile container, to produce a saturated solution in each sterile container. A magnetic stirrer bar was added to each saturated solution and the container placed on a magnetic stirrer at rt for approximately 72 hrs. Each saturated solution was centrifuged, and the supernatants filtered through a 0.22 μm PVDF filter. The first 1 mL of filtrate was discarded.

The three separate resulting filtered solutions were equivalent to approximately 100 mg/mL. One of the resulting filtered solutions was retained to be used as the 100 mg/mL dose formulation.

6 mL from the second of the three 100 mg/mL resultant filtered solutions was diluted with 14 mL of the filtered vehicle to produce a 30 mg/mL filtered solution.

2 mL from the third of the three 100 mg/mL resultant filtered solutions was diluted with 18 mL of the filtered vehicle to produce a 10 mg/mL filtered solution.

Formulated Test Article Sampling and Analysis

On the day of dose administration (Day 1), duplicate 1mL samples were obtained from the top, middle and bottom strata of each Compound 1 formulation concentration. All dose formulation samples were refrigerated (2 to 8° C.) prior to analysis. One set of samples of each Compound 1 formulation concentration were analyzed for concentration and homogeneity of the test article in the vehicle. The other set of duplicate samples were stored refrigerated (2 to 8° C.) until the results were obtained and deemed acceptable.

Concentration results were 90.4 to 105% of label claim.

Pharmacokinetic Study

Fifteen experimentally nave male Dutch-belted rabbits, approximately 7 months old and weighing 1.7 to 2.3 kilograms at the outset of the study were assigned to treatment groups as shown in Table 1 below.

TABLE 1
Group Identification

| | Dose | Dose | Dosing | Number of |
|---|---|---|---|---|
| | Level* | Volume | Concentration* | Animals |
| Group | (μg/eye) | (mL/eye) | (μg/mL) | Males |
| 1. Compound 1 | 0.5 | 0.05 | 10 | 5 |
| Low-dose | | | | |
| 2. Compound 1 | 1.5 | 0.05 | 30 | 5 |
| Mid-dose | | | | |
| 3. Compound 1 | 5 | 0.05 | 100 | 5 |
| High-dose | | | | |

*Concentrations and dose levels were expressed as free base form of the test article.

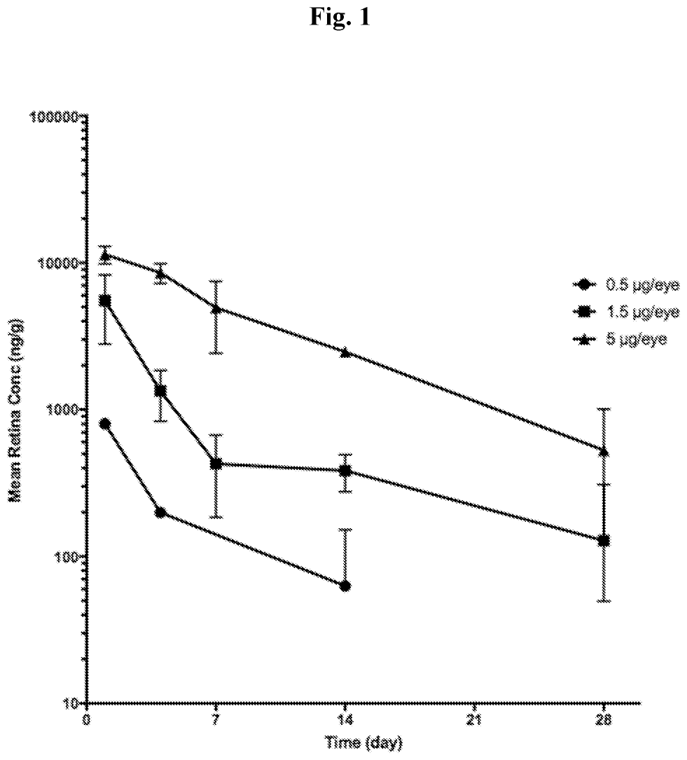

Animals were administered a single bilateral, intravitreal injection of the Compound 1 aqueous solution pharmaceutical formulations once on Day 1. At the scheduled terminal sacrifices on Days 2, 5, 8, 15 and 29, one animal/group in Groups 1-3 had both eyes harvested at necropsy and dissected to isolate the following for analysis of Compound 1 concentration: aqueous humor, vitreous humor and choroid/retina (or retina-choroid tissue complex). The retinal and choroid tissue concentrations are shown in FIG. 1. The AUC, Cmax and t_{1/2} data are summarized in Tables 5, 6 and 7 respectively.

Example 2

Formulation of 100 μg/mL solution of Compound 1 in 9.8% trehalose, 0.03% histidine (2mM), in

SWFI was prepared and dosed at 5 μg/eye at monthly intervals. Ocular tissue and fluid concentrations of Compound 1 were characterized following multiple bilateral intravitreal injections to both eyes of Dutch Belted rabbits, or a single eye of cynomolgus monkeys. Further details are provided below.

Preparation of 10, 30, 100 and 300 μg/mL Solution Formulations of Compound 1

A 9.8% w/w trehalose and 2 mM histidine buffer solution is prepared by dissolving L-histidine (1.09 g) and trehalose dihydrate (356.7 g) in SWFI (3270 g) with agitation. The buffer pH is adjusted using 1.0 N HCl solution as needed and diluted to 3640 g with SWFI to yield the buffer solution. Compound 1 (0.340 g) is dissolved in the trehalose-histidine buffer (2800 g) solution with high energy rotor stator mixing at 40° C. for sufficient time to provide a visibly clear, colorless solution, approximately 15-30 min. The pH of the solution is adjusted as needed with 1.0 N HCl solution. HPLC is used to determine concentration of Compound 1 in the solution and the solution is diluted as needed with the trehalose-histidine buffer solution. The resulting 100 μg/mL solution formulation of Compound 1 is sterile filtered through two PVDF sterile filtration modules in series into a sterile, depyrogenated pyrex glass container.

10, 30 and 300 μg/mL solution formulations of Compound 1 were prepared analogously with a common buffer and with the amount of Compound 1 being varied. For example, 0.104 g of Compound 1 was used to prepare the 30 μg/mL solution and 0.0363 g of Compound 1 was used to prepare the 10 μg/mL solution formulations.

Table 2 below provides analytical and characterization data for the 10, 30, 100 and 300 μg/mL solution formulations of Compound 1.

TABLE 2
Analytical and characterization data for the 10, 30,
100 and 300 μg/mL solution formulations of Compound 1

| | | 10 μg/mL | 30 μg/mL | 100 μg/mL | 300 μg/mL |
|---|---|---|---|---|---|
| Appearance* | | C, C, L, FVP | C, C, L, FVP | C, C, L, FVP | C, C, L, FVP |
| Assay (% LC)** | | 108 | 106 | 103 | 107 |
| Purity (area %) | | 99.9 | 99.9 | 100 | 100 |
| Impurities*** | | RRT 0.64-0.17% | RRT 0.57-0.11% | ND | ND |
| pH | | 5.8 | 5.5 | 5.5 | 5.6 |
| Osmolality (mOsmol/Kg) | | 304 | 302 | 303 | 307 |
| Particulate | ≥10 μm | 0.1 | 0.1 | 0.1 | 0.4 |
| matter/mL | ≥25 μm | 0.0 | 0.1 | 0.0 | 0.15 |
| | ≥50 μm | 0.0 | 0.0 | 0.0 | 0.0 |

| | | | | |
|---|---|---|---|---|
| Bacterial endotoxin | <0.0500 | <0.0500 | <0.0500 | <0.0500 |
| (EU/mL) | | | | |
| Sterility | Sterile | Sterile | Sterile | Sterile |

*C, C, L, FVP = Clear, Colorless, Liquid, Free from Visible Particles
**% LC = % Label Claim
***ND = not detected

The 10, 30, 100 and 300 mg/mL solution formulations of Compound 1 are stable when filled into 2 mL clear type 1 glass vials sealed with chlorobutyl rubber stoppers, as shown by the data in Table 3.

TABLE 3
Stability data for the 10, 30, 100 and 300 ug/mL solution formulations of Compound 1

| | | 10 μg/mL | | 30 μg/mL | | 100 μg/mL | | 300 μg/mL | |
|---|---|---|---|---|---|---|---|---|---|
| | | | 36 months | | 36 months | | 36 months | | 36 months |
| | | | at 25° C. | | at 25° C. | | at 25° C. | | at 25° C. |
| | | | and 60% | | and 60% | | and 60% | | and 60% |
| | | Initial | RH⁺ | Initial | RH⁺ | Initial | RH⁺ | Initial | RH⁺ |
| Appearance* | | C, C, L, | C, C, L, | C, C, L, | C, C, L, | C, C, L, | C, C, L, | C, C, L, | C, C, L, |
| | | FVP | FVP | FVP | FVP | FVP | FVP | FVP | FVP |
| Assay (% LC)** | | 108 | 109 | 106 | 103 | 103 | 101 | 107 | 106 |
| Purity (area %) | | 99.9 | 99.7 | 99.9 | 99.9 | 100 | 99.8 | 100 | 99.9 |
| Impurities | | | | | | | | | |
| RRT 0.32 | | — | — | — | 0.03% | — | — | — | — |
| RRT 0.56-0.58 | | — | 0.10% | 0.11% | 0.04% | — | 0.04% | — | — |
| RRT 0.59-0.60 | | — | 0.06% | — | — | — | — | — | — |
| RRT 0.63-0.65 | | 0.17% | 0.05% | — | — | — | — | — | — |
| RRT 0.69 | | — | 0.05% | — | 0.07% | — | 0.06% | — | 0.06% |
| RRT 0.83 | | — | — | — | 0.03% | — | — | — | — |
| RRT 1.34 | | — | 0.04% | — | — | — | 0.06% | — | 0.04% |
| RRT 1.39 | | — | — | — | — | — | 0.04% | — | 0.03% |
| pH | | 5.8 | 5.9 | 5.5 | 5.6 | 5.5 | 5.6 | 5.6 | 5.7 |
| Osmolality | | 304 | 307 | 302 | 307 | 303 | 305 | 307 | 308 |
| (mOsmol/Kg) | | | | | | | | | |
| Particulate | ≥10 μm | 0.1 | 0.25 | 0.1 | 0.25 | 0.1 | 0.2 | 0.4 | 0.6 |
| matter/mL | ≥25 μm | 0.0 | 0.2 | 0.1 | 0.2 | 0.0 | 0.2 | 0.15 | 0.55 |
| | ≥50 μm | 0.0 | 0.1 | 0.0 | 0.15 | 0.0 | 0.15 | 0.0 | 0.35 |

| | | | | | | | | |
|---|---|---|---|---|---|---|---|---|
| Bacterial endotoxin | <0.0500 | <0.0500 | <0.0500 | <0.0500 | <0.0500 | <0.0500 | <0.0500 | <0.0500 |
| (EU/mL) | | | | | | | | |
| Sterility | Sterile | Sterile | Sterile | Sterile | Sterile | Sterile | Sterile | Sterile |

*C, C, L, FVP = Clear, Colorless, Liquid, Free from Visible Particles
**% LC = % Label Claim
⁺RH = relative humidity

Ocular Pharmacokinetic Study in Rabbit

Nineteen experimentally nave male Dutch-belted rabbits, approximately 7 months old and weighing 1.7 to 2.3 kilograms at the outset of the study were assigned to treatment groups as shown in Table 4 below.

TABLE 4
Group Identification

| | Dose | Dose | Dosing | Number of |
|---|---|---|---|---|
| | Level* | Volume | Concentration* | Animals |
| Group | (μg/eye) | (mL/eye) | (μg/mL) | Males |
| Compound 1 | 5 | 0.05 | 100 | 19 |

*Concentrations and dose levels were expressed as free base form of the test article

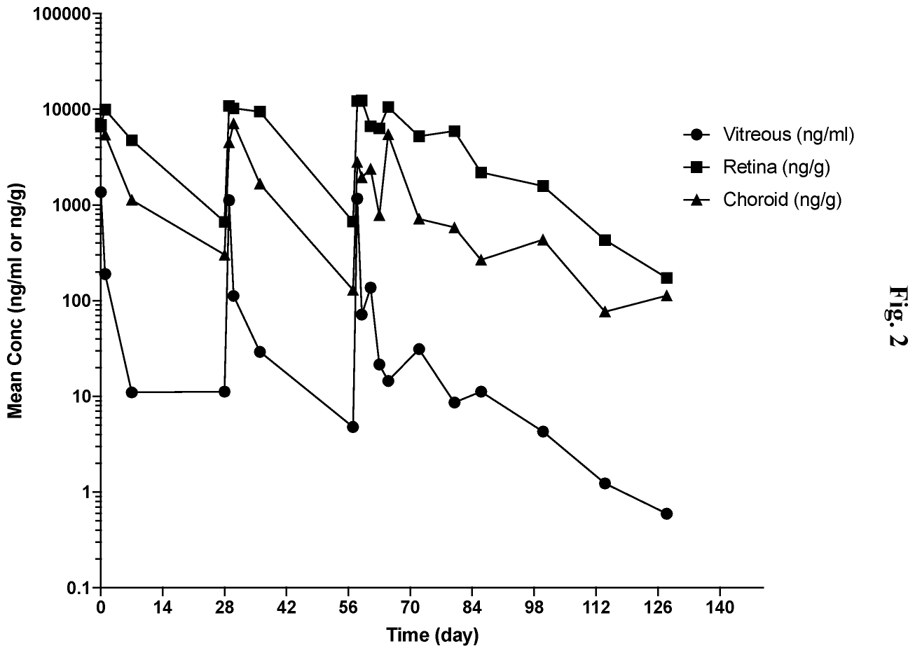

Animals were administered a single bilateral, intravitreal injection of the Compound 1 aqueous solution pharmaceutical formulations once on Day 1, 29 and 57. At the scheduled terminal sacrifices on Days 1, 2, 8, 29, 30, 36, 57, 58, 60, 62, 64, 71, 78, 85, 99, 113 and 127 one animal/group had both eyes harvested at necropsy and dissected to isolate the following for analysis of Compound 1 concentration: aqueous humor, vitreous humor, choroid and retina. The vitreous, retinal and choroid tissue concentrations are shown in FIG. 2. The resulting AUC and Cmax data are summarized in Tables 5 and 6 respectively.

For the purpose of comparison, data from Compound 1 formulated in phosphate buffered saline (4.2 mg/mL of Compound 1), shown in FIG. 2 of WO2013/005045 are also provided.

The ocular tissue (vitreous, retina and choroid) concentrations demonstrate the unexpected long half-life of the active ingredient when delivered as an aqueous solution pharmaceutical composition and the retina (and choroid) levels confirm that the active ingredient was able to reach the posterior ocular tissues.

TABLE 5
Summary of Compound 1 Ocular Tissue and Fluid Exposure (AUC) in Rabbits
after Single, or Multiple, Bilateral IVT Dosing of Compound 1

| | Compound 1 | Duration of | AUC₀₋ₗₐₛₜ (ng · h/mL or ng · h/g) in Ocular Tissue | | |
|---|---|---|---|---|---|
| | IVT Dose | Sampling | | Retina- | |
| | Level | Post Dose | Vitreous | Choroid | Aqueous |
| Composition | (μg/eye) | (days) | Humor | Tissue | Humor |
| Sterile phosphate | 0.21 | 7 | 2,280 | 60,210 | NC |
| buffered saline | | | | | |
| Example 1 | 0.5 | 29 | NC | NC | NC |
| | 1.5 | 29 | NC | 532,000 | NC |
| | 5 | 29 | 2,390 | 2,470,000 | NC |
| Example 2 | 5 | 28* | 33,200 | 4,380,000** | 1,470 |

NC = Not calculated; concentrations below the limit of quantitation of the assay or insufficient data
*AUC for 28 days post third Dose; Sampling interval Day 57-85
**Retina values only

TABLE 6
Summary of Compound 1 Ocular Tissue and Fluid Exposure (Cₘₐₓ) in Rabbits
after Single, or Multiple, Bilateral IVT Dosing of Compound 1

| | Compound 1 | Duration of | Cₘₐₓ (ng/mL or ng/g) in Ocular Tissue | | |
|---|---|---|---|---|---|
| | IVT Dose | Sampling | | Retina- | |
| | Level | Post Dose | Vitreous | Choroid | Aqueous |
| Composition | (μg/eye) | (days) | Humor | Tissue | Humor |
| Sterile phosphate | 0.21 | 7 | 54.2 | 920 | 1.1 |
| buffered saline | | | | | |
| Example 1 | 0.5 | 29 | 1.22 | 803 | NC |
| | 1.5 | 29 | 10.4 | 5540 | NC |
| | 5 | 29 | 24.3 | 11,400 | 0.152 |
| | | 29* | 1,360 | 9,910⁺ | 6.16 |
| Example 2 | 5 | 28** | 1,120 | 10,800⁺ | 7.59 |
| | | 70*** | 5460 | 12,300⁺ | 6.32 |

NC = Not calculated; concentrations below the limit of quantitation of the assay or insufficient data
*Cmax for dose interval Day 1-29,
**Cmax for Dose interval Day 29-57,
***Cmax for Dose interval Day 57-127
⁺Retina values only

TABLE 7
Summary of Compound 1 Ocular Tissue and Fluid Exposure
(terminal half-life, t_{1/2}) in Rabbits after Single,
or Multiple, Bilateral IVT Dosing of Compound 1

| | Compound 1 | Duration of | Terminal half-life (h) | |
|---|---|---|---|---|
| | IVT Dose | Sampling | in Ocular Tissue | |
| | Level | Post Dose | Vitreous | Retina-Choroid |
| Composition | (μg/eye) | (days) | Humor | Tissue |
| Sterile | 0.21 | 7 | NC | 56 |
| phosphate | | | | |
| buffered saline | | | | |
| Example 1 | 0.5 | 29 | NC | NC |
| | 1.5 | 29 | NC | 275 |
| | 5 | 29 | 216 | 155 |

NC = Not calculated; concentrations below the limit of quantitation of the assay or insufficient data

Ocular Pharmacokinetic Study in Monkey

Six experimentally naive male cynomolgus monkeys were assigned to treatment group as shown in Table 8 below.

TABLE 8
Group Identification

| | Dose | Dose | Dosing | Number of |
|---|---|---|---|---|
| | Level* | Volume | Concentration* | Animals |
| Group | (μg/eye) | (mL/eye) | (μg/mL) | Males |
| Compound 1 | 5 | 0.05 | 100 | 6 |

*Concentrations and dose levels were expressed as free base form of the test article

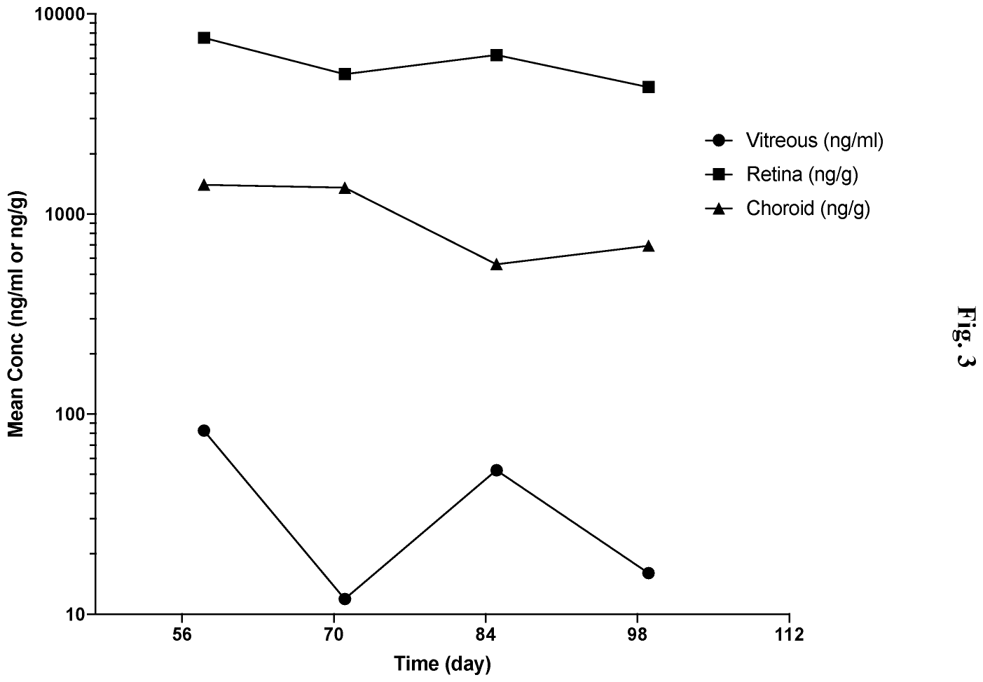

Animals were administered a single bilateral intravitreal injection of the Compound 1 aqueous solution pharmaceutical formulations, once on Day 1, 29 and 57. At scheduled terminal sacrifices on Days 58, 71, 85 and 99 one animal/group had both eyes harvested at necropsy and dissected to isolate the following for analysis of Compound 1 concentration: aqueous humor, vitreous humor, choroid and retina. The resulting AUC and Cmax data are summarized in Table 9. The vitreous, retinal and choroid tissue concentrations are shown in FIG. 3. The ocular tissue (vitreous, retina and choroid) concentrations demonstrate the unexpected long half-life of the active ingredient when delivered as an aqueous solution pharmaceutical composition and the retina (and choroid) levels confirm that the active ingredient was able to reach the posterior ocular tissues.

TABLE 9
Ocular Pharmacokinetics in Pigmented Moneys
over a 99 Day Sampling Period Following a Multiple
Intravitreal Injection of Compound 1

| | | Composite Mean Ocular Tissue | |
|---|---|---|---|
| | | Noncompartmental Parameters | |
| | | Cₘₐₓ (ng/mL or | AUC₀₋ₗₐₛₜ (ng · h/mL |
| Group/IVT | | ng/g) in Ocular | or ng · h/g) in |
| Dose | Fluid or Tissue | Tissue | Ocular Tissue |
| Group 1 | Retina- Tissue | 7,580 | 166,000 |
| 5 μg/eye | Choroid-Tissue | 693 | 32,200 |
| | Vitreous Humor | 82.6 | 1,110 |
| | Aqueous Humor | 92.7 | 1,410 |

AUC 0-last calculated post third Dose; Sampling interval Day 57-99

It will be understood that the invention has been described by way of example only and modifications may be made whilst remaining within the scope and spirit of the invention.

1. A pharmaceutical composition in the form of an aqueous solution for parenteral administration comprising at least one non-ionic tonicity agent, at least one buffer and an active ingredient that is a compound of formula I 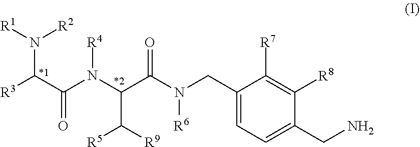
wherein:
R¹ is H, alkyl, —COalkyl, —COaryl, —COheteroaryl, —CO₂alkyl, —(CH₂)ₐOH, —(CH₂)_{b}COOR¹⁰, —(CH₂)_{c}CONH₂, —SO₂alkyl, or —SO₂aryl;
R² is H or alkyl;
R³ is H, alkyl, —(CH₂)_{d}aryl, —(CH₂)eheteroaryl, —(CH₂)fcycloalkyl, —(CH₂)_{g}heterocycloalkyl, —CH(cycloalkyl)₂ or —CH(heterocycloalkyl)₂;
R⁴ and R⁶ areₐ independently, H or alkyl;
R⁵ is scicctcd from H, alkyl, alkoxy or OH;
or R⁴ and R⁵, together with the atoms to which they are attached, may join to form a 5-or 6-membered azacycloalkyl structure;
R⁷ and R⁸ are independently, H, alkyl, alkoxy, CN or halo;
R⁹ is aryl or heteroaryl;
R¹⁰ is H or alkyl;
a, b, c, d, e, f and g are independently 1, 2 or 3;
*1 and *2 denote chiral centres;
alkyl is a linear saturated hydrocarbon having up to 10 carbon atoms (C₁-C₁₀) or a branched saturated hydrocarbon of between 3 and 10 carbon atoms (C₃-C₁₀); wherein the alkyl is optionally substituted with 1 or 2 substituents that are, independently, (C₃-C₁₀)cycloalkyl, (C₁-C₆)alkoxy, OH, CN, CF₃, COOR¹¹, fluoro or NR¹¹R¹²;
cycloalkyl is a mono-or bi-cyclic saturated hydrocarbon of between 3 and 10 carbon atoms; wherein the cycloalkyl is optionally fused to an aryl group;
heterocycloalkyl is a C-linked or N-linked 3 to 10 membered saturated, mono-or bi-cyclic ring, wherein said heterocycloalkyl ring contains, where possible, 1, 2 or 3 heteroatoms that are, independently, N, NR¹¹ or O;
alkoxy is a linear O-linked hydrocarbon of between 1 and 6 carbon atoms (C₁-C₆) or a branched O-linked hydrocarbon of between 3 and 6 carbon atoms (C₃-C₆); wherein the alkoxy is optionally substituted with 1 or 2 substituents that are, independently, (C₃-C₁₀)cycloalkyl, OH, CN, CF₃, COOR¹¹, fluoro or NR¹¹R¹²;
aryl is phenyl, biphenyl or naphthyl; wherein the aryl is optionally substituted with up to 5 substituents that are, independently, alkyl, alkoxy, OH, halo, CN, COOR¹¹, CF₃ or NR¹¹R¹²;
heteroaryl is a 5, 6, 9 or 10 membered mono-or bi-cyclic aromatic ring, containing, where possible, 1, 2 or 3 ring members that are, independently, N, NR¹¹, S or O; wherein the heteroaryl may be is optionally substituted with 1, 2 or 3 substituents that are, independently, alkyl, alkoxy, OH, halo, CN, COOR¹¹, CF₃ or NR¹¹R¹²;
R¹¹ and R¹² are, independently, H alkyl;
or a tautomer, isomer, stereoisomer or a pharmaceutically acceptable salt thereof.

2. The composition of claim 1, wherein R⁹ is phenyl or naphthyl, wherein the phenyl is optionally substituted with up to 3 substituents that are, independently, alkyl, alkoxy, OH, halo, CN, COOR¹¹, CF₃ or NR¹¹R¹².

3. The composition of claim 1, wherein R⁹ is phenyl, 1-naphthalene, 2,4-dichlorophenyl, 3,4-dichlorophenyl, 3,4-difluorophenyl, 4-chlorophenyl, 4-trifluoromethylphenyl or 4-ethoxyphenyl.

4. The composition of claim 1, wherein R¹ is H, —COaryl, —COalkyl, —CH₂COOH, —SO₂Ph or —SO₂CH₃.

5. The composition of claim 1, wherein R¹ is COalkyl or —COaryl.

6. The composition of claim 1, wherein R³ is

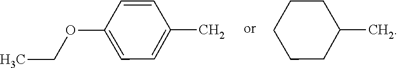

7. The composition of claim 1, wherein R⁴ and R⁶ are or CH_{3.}

8. The composition of claim 1, wherein the stereochemical configuration about chiral centre *1 is R.

9. The composition of claim 1, wherein the stereochemical configuration about chiral centre *2 is S.

10. The composition of claim 1, wherein a is 2 and b, c, d, e, f, g, h, j, l and m are 1.

11. The composition of claim 1, wherein the compound of formula I is:
(S)-N-(4-Aminomethyl-benzyl)-2-[(R)-3-(4-ethoxy-phenyl)-2-propionylamino-propionylamino]-3-phenyl-propionamide;
N-[(R)-1-[(S)-1-(4-Aminomethyl-benzylcarbamoyl)-2-phenyl-ethylcarbamoyl]-2-(4-ethoxy-phenyl)-ethyl]-benzamide;
{(R)-1-[(S)-1-(4-Aminomethyl-benzylcarbamoyl)-2-phenyl-ethylcarbamoyl]-2-cyclohexyl-ethylamino}-acetic acid;
(S)-N-(4-Aminomethyl-3-fluoro-benzyl)-2-[(R)-3-(4-ethoxy-phenyl)-2-propionylamino-propionylamino]-3-phenyl-propionamide;
(S)-N-(4-Aminomethyl-2-chloro-benzyl)-2-[(R)-3-(4-ethoxy-phenyl)-2-propionyl amino-propionylamino]-3-phenyl-propionamide;
(S)-N-(4-Aminomethyl-benzyl)-3-(3,4-di chl oro-phenyl)-2-[(R)-3-(4-ethoxy-phenyl)-2-propionylamino-propionylamino]-propionamide;
(S)-N-(4-Aminomethyl-3-chloro-benzyl)-2-[(R)-3-(4-ethoxy-phenyl)-2-propionyl amino-propionylamino]-3-phenyl-propionamide;
(S)-N-(4-Aminomethyl-benzyl)-2-{[(R)-3-(4-ethoxy-phenyl)-2-propionylamino-propionyl]-methyl-amino}-3-phenyl-propionamide;
({(R)-1-[(S)-1-(4-Aminomethyl-benzylcarbamoyl)-2-phenyl-ethylcarbamoyl]-2-cyclohexyl-ethyl}-methyl-amino)-acetic acid;
(S)-N-(4-Aminomethyl-3-fluoro-benzyl)-2-{[(R)-3-(4-ethoxy-phenyl)-2-propionylamino-propionyl]-methyl-amino}-3-phenyl-propionamide;
N-[(R)-1-{[(S)-1-(4-Aminomethyl-benzylcarbamoyl)-2-phenyl-ethyl]-methyl-carbamoyl}-2-(4-ethoxy-phenyl)-ethyl]-benzamide;
N-[(R)-1-{[(S)-1-(4-Aminomethyl-benzylcarbamoyl)-2-phenyl-ethyl]-methyl-carbamoyl}-2-(4-ethoxy-phenyl)-ethyl]-isobutyramide;
Naphthalene-1-carboxylic acid [(R)-1-[(S)-1-(4-aminomethyl -benzylcarbamoyl)-2-phenyl-ethylcarbamoyl]-2-(4-ethoxy-phenyl)-ethyl]-amide;
N-[(R)-1-[(S)-1-(4-Aminomethyl-benzylcarbamoyl)-2-phenyl-ethyl carb amoyl]-2-(4-ethoxy-phenyl)-ethyl]-4-chloro-benzamide;
N-[(R)-1-[(S)-1-(4-Aminomethyl-benzylcarbamoyl)-2-phenyl-ethyl carb amoyl]-2-(4-ethoxy-phenyl)-ethyl]-2,4-dichloro-benzamide;
N-[(R)-1-[(S)-1-(4-Aminomethyl-benzylcarbamoyl)-2-phenyl-ethylcarbamoyl]-2-(4-ethoxy-phenyl)-ethyl]-3,4-difluoro-benzamide;
(R)-2-Amino-N-[(1S,2S)-1-(4-aminomethyl-benzylcarbamoyl)-2-hydroxy-2-phenyl-ethyl]-3-(4-ethoxy-phenyl)-propionamide;
N-[(R)-1-[(S)-1-(4-Aminomethyl-benzylcarbamoyl)-2-phenyl-ethylcarbamoyl]-2-(4-ethoxy-phenyl)-ethyl]-nicotinamide;
(2 S, 3 S)-N-(4-Aminomethyl-benzyl)-2-[(R)-3-(4-ethoxy-phenyl)-2-propi onyl amino-propionylamino]-3-hydroxy-3-phenyl-propionamide;
N-[(R)-1-[(S)-1-(4-Aminomethyl-benzylcarbamoyl)-2-phenyl-ethylcarbamoyl]-2-(4-ethoxy-phenyl)-ethyl]-isonicotinamide;
Thiophene-3-carboxylic acid-[(R)-1-[(S)-1-(4-aminomethyl-benzylcarbamoyl)-2-phenyl-ethylcarbamoyl]-2-(4-ethoxy-phenyl)-ethyl]-amide;
Thiophene-2-carboxylic acid [(R)-1-[(S)-1-(4-aminomethyl-benzylcarbamoyl)-2-phenyl-ethylcarbamoyl]-2-(4-ethoxy-phenyl)-ethyl]-amide;
Cyclohexanecarboxylic acid [(R)-1-[(S)-1-(4-aminomethyl-benzylcarbamoyl)-2-phenyl-ethylcarbamoyl]-2-(4-ethoxy-phenyl)-ethyl]-amide;
Isoxazole-5-carboxylic acid [(R)-1-[(S)-1-(4-aminomethyl-benzylcarbamoyl)-2-phenyl-ethylcarbamoyl]-2-(4-ethoxy-phenyl)-ethyl]-amide;
Pyridine-2-carboxylic acid [(R)-1-[(S)-1-(4-aminomethyl-benzylcarbamoyl)-2-phenyl-ethylcarbamoyl]-2-(4-ethoxy-phenyl)-ethyl]-amide;
Benzo[b]thiophene-2-carboxylic acid[(R)-1-[(S)-1-(4-aminomethyl-benzylcarbamoyl)-2-phenyl-ethylcarbamoyl]-2-(4-ethoxy-phenyl)-ethyl]-amide;
(R)-N-[(S)-1-(4-Aminomethyl-benzylcarbamoyl)-2-phenyl-ethyl]-2-(4-chloro-benzenesulfonylamino)-3-(4-ethoxy-phenyl)-propionamide;
N-[(R)-1-[(S)-1-(4-Aminomethyl-benzylcarbamoyl)-2-phenyl-ethylcarbamoyl]-2-(4-ethoxy-phenyl)-ethyl]-3-chloro-benzamide;
N-[(R)-1-[(S)-1-(4-Aminomethyl-benzylcarbamoyl)-2-phenyl-ethylcarbamoyl]-2-(4-ethoxy-phenyl)-ethyl]-2-chloro-benzamide
N-[(R)-1-[(S)-1-(4-Aminomethyl-benzylcarbamoyl)-2-phenyl-ethylcarbamoyl]-2-(4-ethoxy-phenyl)-ethyl]-3-trifluoromethyl-benzamide;
N-[(R)-1-[(S)-1-(4-Aminomethyl-benzylcarbamoyl)-2-phenyl-ethylcarbamoyl]-2-(4-ethoxy-phenyl)-ethyl]-4-methyl-benzamide;
N-[(R)-1-[(S)-1-(4-Aminomethyl-benzylcarbamoyl)-2-phenyl-ethylcarbamoyl]-2-(4-ethoxy-phenyl)-ethyl]-3,4-dichloro-benzamide;
N-[(R)-1-[(S)-1-(4-Aminomethyl-benzylcarbamoyl)-2-phenyl-ethylcarbamoyl]-2-(4-ethoxy-phenyl)-ethyl]-4-methoxy-benzamide;
(S)-N-(4-Aminomethyl-benzyl)-2-[(R)-3-(4-ethoxy-phenyl)-2-(2-phenylacetylamino-acetylamino)-propionylamino]-3-phenyl-propionamide;
N-[(R)-1-[(S)-1-(4-Aminomethyl-benzylcarbamoyl)-2-phenyl-ethylcarbamoyl]-2-(4-ethoxy-phenyl)-ethyl]-4-fluoro-benzamide;
N-[(R)-1-[(S)-1-(4-Aminomethyl-benzylcarbamoyl)-2-phenyl-ethylcarbamoyl]-2-(4-ethoxy-phenyl)-ethyl]-6-methyl-nicotinamide;
N-[(R)-1-[(S)-1-(4-Aminomethyl-benzylcarbamoyl)-2-phenyl-ethylcarbamoyl]-2-(4-ethoxy-phenyl)-ethyl]-2-methyl-nicotinamide;
N-[(R)-1-[(S)-1-(4-Aminomethyl-benzylcarbamoyl)-2-phenyl-ethylcarbamoyl]-2-(4-ethoxy-phenyl)-ethyl]-2,6-dichloro-nicotinamide;
N-[(R)-1-[(S)-1-(4-Aminomethyl-benzylcarbamoyl)-2-phenyl-ethylcarbamoyl]-2-(4-ethoxy-phenyl)-ethyl]-5,6-dichloro-nicotinamide;
N-[(R)-1-[(S)-1-(4-Aminomethyl-benzylcarbamoyl)-2-phenyl-ethylcarbamoyl]-2-(4-ethoxy-phenyl)-ethyl]-2,3,6-trifluoro-isonicotinamide;
N-[(R)-1-[(S)-1-(4-Aminomethyl-benzylcarbamoyl)-2-phenyl-ethylcarbamoyl]-2-(4-ethoxy-phenyl)-ethyl]-3,3,3-trifluoro-propionamide;
2,4-Dimethyl-thiazole-5-carboxylic acid [(R)-1-[(S)-1-(4-aminomethyl-benzylcarb amoyl)-2-phenyl-ethylcarbamoyl]-2-(4-ethoxy-phenyl)-ethyl]-amide;
2-Methyl-thiazole-5-carboxylic acid [(R)-1-[(S)-1-(4-aminomethyl-benzylcarbamoyl)-2-phenyl-ethylcarbamoyl]-2-(4-ethoxy-phenyl)-ethyl]-amide;
3-Chloro-thiophene-2-carboxylic acid [(R)-1-[(S)-1-(4-aminomethyl-benzylcarbamoyl)-2-phenyl-ethylcarbamoyl]-2-(4-ethoxy-phenyl)-ethyl]-amide;
4-Methyl-thiazole-5-carboxylic acid[(R)-1-[(S)-1-(4-aminomethyl-benzylcarbamoyl)-2-phenyl-ethylcarbamoyl]-2-(4-ethoxy-phenyl)-ethyl]-amide;
Furan-2-carboxylic acid [(R)-1-[(S)-1-(4-aminomethyl-benzylcarbamoyl)-2-phenyl-ethylcarbamoyl]-2-(4-ethoxy-phenyl)-ethyl]-amide;
3-Methyl-thiophene-2-carboxylic acid [(R)-1-[(S)-1-(4-aminomethyl-benzylcarbamoyl)-2-phenyl-ethylcarbamoyl]-2-(4-ethoxy-phenyl)-ethyl]-amide;
N-[(R)-1-[(S)-1-(4-Aminomethyl-benzylcarbamoyl)-2-phenyl-ethylcarbamoyl]-2-(4-ethoxy-phenyl)-ethyl]-2-methoxy-isonicotinamide;
3-Methyl-1H-pyrrole-2-carboxylic acid [(R)-1-[(S)-1-(4-aminomethyl-benzylcarbamoyl)-2-phenyl-ethylcarbamoyl]-2-(4-ethoxy-phenyl)-ethyl]-amide;
3-Amino-thiophene-2-carboxylic acid [(R)-1-[(S)-1-(4-aminomethyl-benzylcarbamoyl)-2-phenyl-ethylcarbamoyl]-2-(4-ethoxy-phenyl)-ethyl]-amide;
N-[(R)-1-[(S)-1-(4-Aminomethyl-benzylcarbamoyl)-2-phenyl-ethylcarbamoyl]-2-(4-propoxy-phenyl)-ethyl]-benzamide;
N-[(R)-1-[(S)-1-(4-Aminomethyl-benzylcarbamoyl)-2-pyridin-2-yl-ethylcarbamoyl]-2-(4-ethoxy-phenyl)-ethyl]-benzamide;
N-[(R)-1-[(S)-1-(4-Aminomethyl-benzylcarbamoyl)-2-(3,4-dichloro-phenyl)-ethylcarbamoyl]-2-(4-ethoxy-phenyl)-ethyl]-benzamide;
N-[(R)-1-[(S)-1-(4-Aminomethyl-benzylcarbamoyl)-2-(4-chloro-phenyl)-ethylcarbamoyl]-2-(4-ethoxy-phenyl)-ethyl]-benzamide;
N-[(R)-1-[(S)-1-(4-Aminomethyl-benzylcarbamoyl)-2-(4-fluoro-phenyl)-ethylcarbamoyl]-2-(4-ethoxy-phenyl)-ethyl]-benzamide;
N-[(R)-1-[(S)-1-(4-Aminomethyl-benzylcarbamoyl)-2-pyridin-3-yl-ethylcarbamoyl]-2-(4-ethoxy-phenyl)-ethyl]-benzamide;
N-[(R)-1-[(S)-1-(4-Aminomethyl-benzylcarbamoyl)-2-(4-methoxy-phenyl)-ethylcarbamoyl]-2-(4-ethoxy-phenyl)-ethyl]-benzamide;
N-[(R)-1-[(S)-1-(4-Aminomethyl-benzylcarbamoyl)-2-pyridin-4-yl-ethylcarbamoyl]-2-(4-ethoxy-phenyl)-ethyl]-benzamide;
N-[(R)-1-[(S)-1-(4-Aminomethyl-benzylcarbamoyl)-2-(3-fluoro-phenyl)-ethylcarbamoyl]-2-(4-ethoxy-phenyl)-ethyl]-benzamide;
N-[(R)-1-[(S)-1-(4-Aminomethyl-benzylcarbamoyl)-2-thiophen-2-yl-ethylcarbamoyl]-2-(4-ethoxy-phenyl)-ethyl]-benzamide;
N-[(R)-1-[(S)-1-(4-Aminomethyl-benzylcarbamoyl)-2-thiophen-3-yl-ethylcarbamoyl]-2-(4-ethoxy-phenyl)-ethyl]-benzamide;
N-[(R)-1-[(S)-1-(4-Aminomethyl-benzylcarbamoyl)-2-thiazol-4-yl-ethylcarbamoyl]-2-(4-ethoxy-phenyl)-ethyl]-benzamide;
N-[(R)-1-[(S)-1-(4-Aminomethyl-benzylcarbamoyl)-2-benzo[b]thiophen-3-yl-ethylcarbamoyl]-2-(4-ethoxy-phenyl)-ethyl]-benzamide;
N-[(R)-1-[(S)-1-(4-Aminomethyl-3-fluoro-benzylcarbamoyl)-2-phenyl-ethylcarbamoyl]-2-(4-ethoxy-phenyl)-ethyl]-benzamide;
N-[(R)-1-[(S)-1-(4-Aminomethyl-3-chloro-benzylcarbamoyl)-2-phenyl-ethylcarbamoyl]-2-(4-ethoxy-phenyl)-ethyl]-benzamide;
Pyridine-2-carboxylic acid [(R)-1-[(S)-1-(4-aminomethyl-benzylcarbamoyl)-2-thiophen-2-yl-ethylcarbamoyl]-2-(4-ethoxy-phenyl)-ethyl]-amide;
N-[(R)-1-[(S)-1-(4-Aminomethyl-benzylcarbamoyl)-2-pyridin-2-yl-ethylcarbamoyl]-2-(4-ethoxy-phenyl)-ethyl]-4-methoxy-benzamide;
Pyridine-2-carboxylic acid [(R)-1-[(S)-1-(4-aminomethyl-3-chloro-benzylcarbamoyl)-2-phenyl-ethylcarbamoyl]-2-(4-ethoxy-phenyl)-ethyl]-amide;
N-[(R)-1-[(S)-1-(4-Aminomethyl-benzylcarbamoyl)-2-pyridin-3-yl-ethylcarbamoyl]-2-(4-ethoxy-phenyl)-ethyl]-4-methoxy-benzamide;
N-[(R)-1-[(S)-1-(4-Aminomethyl-benzylcarbamoyl)-2-(3,4-difluoro-phenyl)-ethylcarbamoyl]-2-(4-ethoxy-phenyl)-ethyl]-isonicotinamide;
Thiophene-2-carboxylic acid [(R)-1-[(S)-1-(4-aminomethyl-benzylcarbamoyl)-2-pyridin-3-yl-ethylcarbamoyl]-2-(4-ethoxy-phenyl)-ethyl]-amide;
N-[(R)-1-[(S)-1-(4-Aminomethyl-benzylcarbamoyl)-2-pyridin-2-yl-ethylcarbamoyl]-2-(4-ethoxy-phenyl)-ethyl]-4-chloro-benzamide;
N-[(R)-1-[(S)-1-(4-Aminomethyl-benzylcarbamoyl)-2-pyridin-2-yl-ethylcarbamoyl]-2-(4-ethoxy-phenyl)-ethyl]-4-methyl-benzamide;
Pyridine-2-carboxylic acid [(R)-1-[(S)-1-(4-aminomethyl-benzylcarbamoyl)-2-(3,4-dichloro-phenyl)-ethylcarbamoyl]-2-(4-ethoxy-phenyl)-ethyl]-amide;
(R)-N-[(S)-1-(4-Aminomethyl-benzylcarbamoyl)-2-pyridin-2-yl-ethyl]-3-(4-ethoxy-phenyl)-2-propionylamino-propionamide;
N-[(R)-1-[(S)-1-(4-Aminomethyl-3-fluoro-benzylcarbamoyl)-2-phenyl-ethylcarbamoyl]-2-(4-ethoxy-phenyl)-ethyl]-isonicotinamide;
Pyridine-2-carboxylic acid [(R)-1-[(S)-1-(4-aminomethyl-3-fluoro-benzylcarbamoyl)-2-phenyl-ethylcarbamoyl]-2-(4-ethoxy-phenyl)-ethyl]-amide;
Thiophene-2-carboxylic acid [(R)-1-[(S)-1-(4-aminomethyl-benzylcarbamoyl)-2-(3,4-dichloro-phenyl)-ethylcarbamoyl]-2-(4-ethoxy-phenyl)-ethyl]-amide;
(R)-N-[(S)-1-(4-Aminomethyl-benzylcarbamoyl)-2-pyridin-3-yl-ethyl]-3-(4-ethoxy-phenyl)-2-propionylamino-propionamide;
N-[(R)-1-[(S)-1-(4-Aminomethyl-benzylcarbamoyl)-2-(3,4-dichloro-phenyl)-ethylcarbamoyl]-2-(4-ethoxy-phenyl)-ethyl]-isonicotinamide;
N-[(R)-1-[(S)-1-(4-Aminomethyl-benzylcarbamoyl)-2-(3,4-dichloro-phenyl)-ethylcarbamoyl]-2-(4-ethoxy-phenyl)-ethyl]-3,3,3-trifluoro-propionamide;
N-[(R)-1-[(S)-1-(4-Aminomethyl-benzylcarbamoyl)-2-pyridin-3-yl-ethylcarbamoyl]-2-(4-ethoxy-phenyl)-ethyl]-4-chloro-benzamide;
Isoxazole-5-carboxylic acid [(R)-1-[(S)-1-(4-aminomethyl-benzylcarbamoyl)-2-pyridin-3-yl-ethylcarbamoyl]-2-(4-ethoxy-phenyl)-ethyl]-amide;
N-[(R)-1-[(S)-1-(4-Aminomethyl-benzylcarbamoyl)-2-pyridin-3-yl-ethylcarbamoyl]-2-(4-ethoxy-phenyl)-ethyl]-4-methyl-benzamide;
N-[(R)-1-[(S)-1-(4-Aminomethyl-benzylcarbamoyl)-2-(3,4-difluoro-phenyl)-ethylcarbamoyl]-2-(4-ethoxy-phenyl)-ethyl]-benzamide;
3-Chloro-thiophene-2-carboxylic acid [(R)-1-[(S)-1-(4-aminomethyl-benzylcarbamoyl)-2-pyridin-3-yl-ethylcarbamoyl]-2-(4-ethoxy-phenyl)-ethyl]-amide;
N-[(R)-1-[(S)-1-(4-Aminomethyl-benzylcarbamoyl)-2-(1H-indol-3-yl)-ethylcarbamoyl]-2-(4-ethoxy-phenyl)-ethyl]-benzamide;
N-[(R)-1-[(S)-1-(4-Aminomethyl-benzylcarbamoyl)-2-benzo[b]thiophen-3-yl-ethylcarbamoyl]-2-(4-ethoxy-phenyl)-ethyl]-isonicotinamide;
3-Acetylamino-thiophene-2-carboxylic acid-[(R)-1-[(S)-1-(4-aminomethyl-benzylcarbamoyl)-2-phenyl-ethylcarbamoyl]-2-(4-ethoxy-phenyl)-ethyl]-amide;
N-[(R)-1-[(S)-1-(4-Aminomethyl-benzylcarbamoyl)-2-(2-fluoro-phenyl)-ethylcarbamoyl]-2-(4-ethoxy-phenyl)-ethyl]-benzamide;
3-Methyl-thiophene-2-carboxylic acid [(R)-1-[(S)-1-(4-aminomethyl-benzylcarbamoyl)-2-pyridin-3-yl-ethylcarbamoyl]-2-(4-ethoxy-phenyl)-ethyl]-amide;
N-[(R)-1-[(S)-1-(4-Aminomethyl-3-methyl-benzylcarbamoyl)-2-phenyl-ethylcarbamoyl]-2-(4-ethoxy-phenyl)-ethyl]-benzamide;
3-Amino-thiophene-2-carboxylic acid [(R)-1-[(S)-1-(4-aminomethyl-benzylcarbamoyl)-2-thiazol-4-yl-ethylcarbamoyl]-2-(4-ethoxy-phenyl)-ethyl]-amide;
3-Chloro-thiophene-2-carboxylic acid [(R)-1-[(S)-1-(4-aminomethyl-benzylcarbamoyl)-2-thiazol-4-yl-ethylcarbamoyl]-2-(4-ethoxy-phenyl)-ethyl]-amide;
N-[(R)-1-[(S)-1-(4-Aminomethyl-benzylcarbamoyl)-2-thi azol-4-yl-ethylcarbamoyl]-2-(4-ethoxy-phenyl)-ethyl]-4-methyl-benzamide;
3-Methyl-1H-pyrrole-2-carboxylic acid [(R)-1-[(S)-1-(4-aminomethyl-benzylcarbamoyl)-2-b enzo[b]thiophen-3-yl-ethylcarbamoyl]-2-(4-ethoxy-phenyl)-ethyl]-amide;
3-Amino-thiophene-2-carboxylic acid [(R)-1-[(S)-1-(4-aminomethyl-benzylcarbamoyl)-2-thiazol-4-yl-ethylcarbamoyl]-2-(4-ethoxy-phenyl)-ethyl]-amide;
3-Acetylamino-thiophene-2-carboxylic acid [(R)-1-[(S)-1-(4-aminomethyl-benzylcarbamoyl)-2-benzo[b]thiophen-3-yl-ethylcarbamoyl]-2-(4-ethoxy-phenyl)-ethyl]-amide;
N-[(R)-1-[(S)-1-(4-Aminomethyl-benzylcarb amoyl)-2-pyridin-3-yl-ethylcarbamoyl]-2-(4-ethoxy-phenyl)-ethyl]-3-methyl-benzamide;
N-[(R)-1-[(S)-1-(4-Aminomethyl-benzylcarbamoyl)-2-pyridin-3-yl-ethylcarbamoyl]-2-(4-ethoxy-phenyl)-ethyl]-2-methyl-benzamide;
3,5-Dimethyl-1H-pyrrole-2-carboxylic acid [(R)-1-[(S)-1-(4-aminomethyl-benzylcarbamoyl)-2-phenyl-ethylcarbamoyl]-2-(4-ethoxy-phenyl)-ethyl]-amide;
N-[(R)-1-[(S)-1-(4-Aminomethyl-3-methyl-benzylcarbamoyl)-2-pyridin-3-yl-ethylcarbamoyl]-2-(4-ethoxy-phenyl)-ethyl]-benzamide;
3-Acetylamino-thiophene-2-carboxylic acid [(R)-1-[(S)-1-(4-aminomethyl-benzylcarbamoyl)-2-thiophen-3-yl-ethylcarbamoyl]-2-(4-ethoxy-phenyl)-ethyl]-amide;
3-Amino-thiophene-2-carboxylic acid [(R)-1-[(S)-1-(4-aminomethyl-benzylcarbamoyl)-2-benzo[b]thiophen-3-yl-ethylcarbamoyl]-2-(4-ethoxy-phenyl)-ethyl]-amide;
3-Acetylamino-thiophene-2-carboxylic acid [(R)-1-[(S)-1-(4-aminomethyl-benzylcarbamoyl)-2-benzo[b]thiophen-3-yl-ethylcarbamoyl]-2-(4-ethoxy-phenyl)-ethyl]-amide;
3-Chloro-thiophene-2-carboxylic acid [(R)-1-{[(S)-1-(4-aminomethyl-benzylcarbamoyl)-2-phenyl-ethyl]-methyl-carbamoyl}-2-(4-ethoxy-phenyl)-ethyl]-amide;
N-[(R)-1-[(1S,2R)-1-(4-Aminomethyl-benzylcarbamoyl)-2-hydroxy-2-phenyl-ethylcarbamoyl]-2-(4-ethoxy-phenyl)-ethyl]-benzamide;
3-Chloro-thiophene-2-carboxylic acid [(R)-1-[(1S,2R)-1-(4-aminomethyl-benzylcarbamoyl)-2-hydroxy-2-phenyl-ethylcarbamoyl]-2-(4-ethoxy-phenyl)-ethyl]-amide; or
N-{(R,S)-1-[(S)-1-(4-Aminomethyl-benzylcarbamoyl)-2-phenyl-ethylcarbamoyl]-2-[4-(2,2,2-trifluoro-ethoxy)-phenyl]-ethyl}-benzamide;
and pharmaceutically acceptable salts thereof.

12. The composition of claim 1, wherein the compound of formula I is N-[(R)-1-[(S)-1-(4-aminomethyl-benzylcarbamoyl)-2-phenyl-ethylcarbamoyl]-2-(4-ethoxy-phenyl)-ethyl]-benzamide 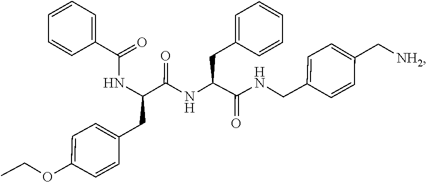 [SMILES: CCOc1ccc(C[C@@H](NC(=O)c2ccccc2)C(=O)N[C@@H](Cc2ccccc2)C(=O)NCc2ccc(CN)cc2)cc1]
or a pharmaceutically acceptable salt thereof.

13. The composition of claim 1, wherein the compound of formula I is present in an amount from about 5 μg/mL to about 300 μg/mL based on the concentration of the free base of the compound of formula I in the composition.

14. The composition of claim 1, wherein the at least one non-ionic tonicity agent is glycerine; a sugar; or a hydroxyethyl starch.

15. The composition of claim 1, wherein the at least one non-ionic tonicity agent is dextrose or trehalose.

16. The composition of claim 1, wherein the at least one non-ionic tonicity agent is present in an amount from about 0.1% to about 30% by weight of the compositions.

17. The composition of claim 1, wherein the composition has a pH of from about 2 to about 10.

18. The composition of claim 1, wherein the composition has an osmolality of from about 250 to about 350 mOsmol/kg.

19. The composition of claim 1, wherein the at least one buffer is histidine, acetate, citrate, cacodylate, bis-tris, maleate, piperazine, IVIES (2-(N-morpholino)ethanesulfonic acid), tartrate, lactate; succinate; sulfate; phosphate; alanine; imidazole; arginine or asparagine.

20. The composition of claim 1, wherein the at least one buffer is histidine.

21. The composition of claim 1, wherein the composition is substantially free of saline.

22. The composition of claim 1, wherein the composition contains histidine as the buffer and dextrose or trehalose as the non-ionic tonicity agent.

23. The composition of claim 1, wherein the composition is substantially free of phosphate buffered saline.

24. The composition of claim 1 further comprising a non-ionic surfactant, wherein the non-ionic surfactant is a polyoxyethylenesorbitan fatty acid ester.

25. The composition of claim 1, wherein the composition is substantially free of a non-ionic surfactant.

26. The composition of claim 1, for injection into the eye.

27. The composition of claim 1, wherein the active ingredient is an HCl salt.

28. The composition of claim 1, wherein the active ingredient has a half-life, t_{1/2}, in vitreous humor of at least about 1 day.

29-30. (canceled)

31. The composition of claim 1, wherein the active ingredient has a half-life, t_{1/2}, in the retina of at least about 1 day.

32-34. (canceled)

35. The composition of claim 1, wherein the active ingredient has a t_{1/2} in the retina-choroid complex of less than about 40 days.

36. (canceled)

37. A method for treating a disease or condition mediated by plasma kallikrein comprising parenteral administration of a pharmaceutical composition of claim 1 to a mammal.

38. The method of claim 37, wherein the disease or condition mediated by plasma kallikrein is impaired visual acuity, diabetic retinopathy, retinal vascular permeability associated with diabetic retinopathy, diabetic macular edema, retinal vascular occlusion, hereditary angioedema, diabetes, pancreatitis, cerebral haemorrhage, cerebral haemorrhage in hyperglycemic patients, nephropathy, cardiomyopathy, neuropathy, inflammatory bowel disease, arthritis, inflammation, septic shock, hypotension, cancer, adult respiratory distress syndrome, disseminated intravascular coagulation, blood coagulation during cardiopulmonary bypass surgery or bleeding from post-operative surgery.

39. The method of claim 37, wherein the disease or condition mediated by plasma kallikrein is impaired visual acuity, diabetic retinopathy, retinal vascular permeability associated with diabetic retinopathy, diabetic macular edema, or retinal vascular occlusion.

40. The method of claim 37, wherein the disease or condition mediated by plasma kallikrein is retinal vascular permeability associated with diabetic retinopathy or diabetic macular edema.

41. The method of claim 37, wherein the disease or condition is a microvascular complication of a disease state.

42-43. (canceled)

44. The method of claim 37, wherein the composition is administered from about once every two weeks to about once every 6 months.

45. The method of claim 37, wherein the method involves combination therapy.

46. The method of claim 45, wherein the method involves laser treatment of the retina.

47. A method for preparing the composition of claim 1, comprising:
(i)(a) preparing a solution of the at least one non-ionic tonicity agent and the at least one buffer in water; and (b) dissolving the compound of formula I, or a pharmaceutically acceptable salt thereof, in the solution prepared in step (a); or
(ii) adding water to a non-aqueous formulation comprising the at least one non-ionic tonicity agent, the at least one buffer and the active ingredient.

48-52. (canceled)

53. The composition of claim 1, wherein the at least one non-ionic tonicity agent is trehalose.
